(19) United States
(12) Patent Application Publication
(10) Pub. No.: US 2011/0269834 A1
(43) Pub. Date: Nov. 3, 2011

(54) COMPOUNDS AND METHODS FOR TREATING RESPIRATORY DISEASES

(75) Inventors: Arun K. Ghosh, West Lafayette, IN (US); Jun Takayama, West Lafayette, IN (US); Andrew David Mesecar, Chicago, IL (US); Michael E. Johnson, Chicago, IL (US); Kiira M. Ratia, Chicago, IL (US); Rima Chaudhuri, Chicago, IL (US); Debbie C. Mulhearn, Chicago, IL (US)

(73) Assignees: THE BOARD OF TRUSTEES OF THE UNIVERSITY OF ILLINOIS, Urbana, IL (US); PURDUE RESEARCH FOUNDATION, West Lafayette, IN (US)

(21) Appl. No.: 13/059,916

(22) PCT Filed: Aug. 21, 2009
(86) PCT No.: PCT/US09/54657

§ 371 Date: Jun. 2, 2011

Related U.S. Application Data
(60) Provisional application No. 61/090,759, filed on Aug. 21, 2008.

(51) Int. Cl. A61K 31/24; C07C 255/58; A61P 31/12; C07C 229/42; A61K 31/166; A61K 31/197; C07C 233/65; C07C 205/45

(52) U.S. Cl. 514/535; 564/180; 564/166; 558/422; 560/20; 562/444; 514/617; 514/622; 514/619; 514/620; 514/567

(57) ABSTRACT

Described herein are compounds and compositions, and methods for using the compounds and compositions, for treating respiratory diseases and illness, such as severe acute respiratory syndrome (SARS).

### CROSS REFERENCE TO RELATED APPLICATIONS

This patent application claims priority to and the benefit of U.S. Provisional Patent Application Ser. No. 61/090,759, filed Aug. 21, 2008, the disclosure of which is incorporated herein by reference.

### TECHNICAL FIELD

This invention pertains to compounds and compositions useful for the treatment respiratory diseases and illness, such as severe acute respiratory syndrome (SARS), and methods of using the compounds and compositions.

### SUMMARY AND BACKGROUND

The first pandemic of the 21st century, the outbreak of the coronavirus that caused severe acute respiratory syndrome (SARS-CoV), emphasizes the continued, global need for developing defenses against emerging infectious agents, particularly those harbored in animals and capable of acquiring the ability to infect humans.

Although the spread of SARS-CoV, which caused the pandemic of 2002-2003, was effectively halted within a few months after the initial outbreaks, the recent isolation of strains from zoonotic origins thought to be the reservoir for SARS-CoV accentuates the possibility of future re-transmissions of SARS-CoV, or related coronaviruses, from animals to humans (Li W, et al. (2005) Bats are natural reservoirs of SARS-like coronaviruses. Science 310(5748):676-679; Lau S K, et al. (2005) Severe acute respiratory syndrome coronavirus-like virus in Chinese horseshoe bats. Proc Natl Acad Sci USA 102(39):14040-14045). The previously referenced publication, and all subsequently referenced publications, are incorporated herein by reference in their entirety. The development of novel antivirals against SARS-CoV is therefore an important safeguard against future outbreaks and pandemics but so far potent antivirals against SARS-CoV with efficacy in animal models have not yet been developed.

However, due to the complex nature of SARS-CoV replication, a number of processes are considered essential to the coronaviral lifecycle and therefore provide a significant number of targets for inhibiting viral replication. An early and essential process is the cleavage of a multidomain, viral polyprotein into 16 mature components termed non-structural proteins (nsps), which assemble into complexes to execute viral RNA synthesis (reviewed in Ziebuhr J (2008) Chapter 5: Coronavirus replicative proteins. Nidoviruses, eds. Perlman S, Gallagher T, & Snijder E J (ASM press, Washington, D.C.), pp 65-82 and Ziebuhr J (2005) The coronavirus replicase. Curr Top Microbiol Immunol 287:57-94). Two cysteine proteases that reside within the polyprotein, a papain-like protease (PLpro) and a 3C-like protease (3CLpro), catalyze their own release and that of the other nsps from the polyprotein, thereby initiating virus-mediated RNA replication. PLpro cleaves the SARS-CoV ORF1a/1ab at three locations to release itself (nsp3), and also nsp1, nsp2, and the remainder of the polypeptide that is subsequently cleaved by 3CLpro. 3CLpro cleaves the polypeptide in 11 locations to release itself (nsp5), along with nsp4, nsp6-11, Pol (nsp12), Hel (nsp13), and nsp14-16. Without being bound by theory, it is believed herein that the recognition sequence for PLpro consists of a four amino acid sequence consisting of a leucine residue attached to two glycine residues via a fourth variable residue, corresponding to P₄, P₂, and P₁, respectively. The following PLpro polyprotein cleavage sites have been reported

| | |
|---|---|
| P₆ P₅ P₄ P₃ P₂ P₁--P₁′ P₂′P₃′ P₄′ | |
| nsp1 . . . R E L N G G--A V T R . . . nsp2 | (SEQ ID NO: 1) |
| nsp2 . . . F R L K G G--A P I K . . . nsp3 | (SEQ ID NO: 2) |
| nsp3 . . . I S L K G G--K I V S . . . nsp4 | (SEQ ID NO: 3) |

Despite numerous biochemical, structural and inhibitor development studies directed at 3CLpro (reviewed in Yang H, Bartlam M, & Rao Z (2006) Drug design targeting the main protease, the Achilles' heel of coronaviruses. Curr Pharm Des 12(35):4573-4590), potent antivirals that directly target 3CLpro have yet to be developed. In contrast, structural and functional studies directed at PLpro are far less numerous but have established important roles for PLpro beyond viral peptide cleavage including deubiquitination, deISGylation, and involvement in virus evasion of the innate immune response (Devaraj S G, et al. (2007) Regulation Of IRF-3-Dependent Innate Immunity By The Papain-Like Protease Domain Of The Severe Acute Respiratory Syndrome Coronavirus. J Biol Chem 282(44):32208-32221; Lindner H A, et al. (2005), The Papain-Like Protease From The Severe Acute Respiratory Syndrome Coronavirus Is A Deubiquitinating Enzyme. J Virol 79(24):15199-15208; Ratia K, et al. (2006) Severe Acute Respiratory Syndrome Coronavirus Papain-Like Protease: Structure Of A Viral Deubiquitinating Enzyme. Proc Natl Acad Sci USA 103(15):5717-5722; Barretto N, et al. (2005) The papain-like protease of severe acute respiratory syndrome coronavirus has deubiquitinating activity. J Virol 79(24):15189-15198; Sulea T, Lindner H A, Purisima E O, & Menard R (2005) Deubiquitination, A New Function Of The Severe Acute Respiratory Syndrome Coronavirus Papain-Like Protease? J Virol 79(7):4550-4551). Recent studies have also shown that an enzyme homologous to PLpro from the human coronavirus 229E, PLP2, is essential for viral replication (Ziebuhr J, et al. (2007) Human Coronavirus 229E Papain-Like Proteases Have Overlapping Specificities, But Distinct Functions In Viral Replication. J Virol 81(8):3922-3932).

The papain-like protease from SARS-CoV (PLpro), has been reported to be essential for viral replication. This protease is not only responsible for processing the viral polyprotein into its functional units, but it also plays a significant role in helping SARS-CoV evade the human immune system. It is believed herein that inhibition of SARS-CoV PLpro will lead to treatment of this devastating disease.

Generally, proteolytic enzymes have been reported to be key regulators of physiological processes in humans and also essential for the replication of pathogenic viruses, parasites and bacteria that cause infectious disease. Their importance in such fundamental processes has been widely recognized and as a result, since the mid-1990s, over 30 new protease inhibitors have entered the marketplace for the treatment of a wide spectrum of diseases including HIV/AIDS (see, e.g., Turk B (2006) Targeting Proteases: Successes, Failures And Future Prospects. Nat Rev Drug Discov 5(9):785-799). These inhibitors target at least 10 structurally-diverse proteases representing every class of protease (metallo, aspartic, serine and threonine) with the exception of the cysteine proteases (Leung D, Abbenante G, & Fairlie D P (2000) Protease Inhibitors: Current Status And Future Prospects. J Med Chem 43(3):305-341).

Historically, the development of cysteine protease inhibitors with drug-like properties has been slowed by a number of challenges, most notable being their toxicity and lack of specificity due to covalent modification of untargeted cysteine residues. As a result, only a small number have entered into late-phase clinical trials thus far. Despite such challenges, cysteine proteases hold significant promise as drug targets since they are involved in many disease-related processes and as such, a number of compounds have entered into preclinical evaluation or development (Leung-Toung R, Li W, Tam T F, & Karimian K (2002) Thiol-dependent enzymes and their inhibitors: a review. Curr Med Chem 9(9):979-1002).

Described herein is the discovery and optimization of a non-covalent inhibitor of the SARS-CoV papain-like protease (PLpro) from the coronavirus that causes SARS. In addition, use of the deubiquinating (DUB) activity of PLpro is described. In particular, compounds that inhibit SARS-CoV viral replication in Vero E6 cells are described, and include examples that inhibit with an EC₅₀ of 15 μM, and importantly display little or no accompanying cytotoxicity. Without being bound by theory, and based on the X-ray structure of PLpro in complex with compounds described herein, it believed herein that the compounds have a unique mode of inhibition whereby they bind within the P4-P3 subsite of the enzyme. In addition, but without being bound by theory, it is believed herein that the compounds described herein induce a conformational change that renders the active site non-functional induce. More particularly, it is believed herein that the conformational change is a loop closure that shuts down catalysis at the active site. The potent inhibition coupled with the binding orientations and subsequent observations demonstrate that PLpro is a viable target for antivirals directed against SARS-CoV, and that potent, non-covalent cysteine protease inhibitors can be developed with specificity directed toward pathogenic, deubiquitinating enzymes (DUBs) without inhibiting host DUBs. Such compounds are useful for treating SARS and other respiratory diseases.

### BRIEF DESCRIPTION OF THE DRAWINGS

FIG. 1. The replicate plot shows the percent inhibition of PLpro by all compounds screened. The hit zone for the assay (>35% inhibition) is indicated by the box. For example, the activity of example compound 1 is shown as a solid circle in the box and labeled (A).

FIG. 2. PLpro inhibitors described herein have antiviral activity against SARS coronavirus. (B) SARS-CoV infected (open circles, lower trace) and mock-infected (solid circles, upper trace) Vero E6 cells were incubated in the presence of inhibitor compounds 1, 5h, 25, or 24 at the concentrations indicated for 48 hours. Cell viability was measured 48 hours post infection using the CellTiter-Glo Luminescent Cell Viability Assay (Promega) and output was expressed as relative luciferase units (RLU). The error bars represent the standard deviation between triplicate samples.

FIG. 3. Inhibitors described herein are competitive and reversible but lead to enzyme inactivation. (A) A Lineweaver-Burk plot shows PLpro activity with the substrate ISG15-AMC at 4 different concentrations of compound 24, which are indicated in the legend of the plot. Data are shown fit to a model representing competitive inhibition (dashed lines) with the following parameters: Kᵢ=0.49 ±0.08 μM, Vₘₐₓ=363±16 min⁻¹, Kₘ=2.4±0.3 μM.

FIG. 4. (B) A graph is shown indicating the percent enzymatic activity regained following a 1 h incubation with selected inhibitors compared to control and the subsequent 3 h dialysis to remove inhibitor. Percent activity was calculated relative to a control sample containing 2% dimethyl sulfoxide (DMSO), but no inhibitor. Undialyzed samples were incubated for the 3 h required for dialysis, and all samples were assayed for activity at the same time. Undialyzed samples are shown as black bars; dialyzed samples are shown as white bars.

### DETAILED DESCRIPTION

It has been discovered herein that the compounds described herein are useful for treating respiratory diseases and illness. Illustrative respiratory diseases and illness treatable with the methods described herein include, but are not limited to, coronavirus-mediated diseases, such as SARS-CoV, HCoV-NL63, and the like, and including SARS, whooping cough, and diseases leading to bronchiolitis, Kawasaki disease, chronic croup, and the like. In another embodiment, the illustrative diseases treatable with the methods described herein include, but are not limited to, diseases caused by at least one pathogen or virus that utilizes PLpro or an equivalent thereof, where inhibition of the PLpro leads to relief from the corresponding disease, such as SARS, whooping cough, and the like.

In one illustrative embodiment of the invention, methods are described for treating a patient in need of relief from a respiratory viral infection. The methods include the step of administering to the patient a therapeutically effective amount of a compound, or pharmaceutical composition comprising the compound, of formula I

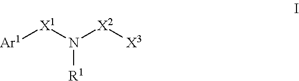

or a pharmaceutically acceptable salt thereof, wherein

Ar¹ is aryl or heteroaryl, each of which is optionally substituted;

X¹ is NR² or CR³R⁴, wherein R² is selected from the group consisting of hydrogen, alkyl, aryl, arylalkyl, heteroaryl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, aryl, arylalkyl and heteroarylalkyl, each of which is optionally substituted; or R³ and R⁴ are taken together with the attached carbon to form a cycloalkylene;

R¹ is hydrogen, alkyl, arylalkyl, heteroarylalkyl, hydroxyl, alkoxyl or a pro-drug moiety, each of which is optionally substituted; and X² is selected from the group consisting of a bond, alkylene and heteroalkylene, or R¹ and X² are taken together with the attached nitrogen to form an optionally substituted heterocycle; and

X³ is an acyl group, a carboxylate group, or a derivative thereof, a sulfonate group, or a sulfonamide group.

In another embodiment, compounds of formula I are described wherein Ar¹ is naphthyl, quinolinyl, isoquinolinyl, or quinazolinyl, each of which is optionally substituted.

In another embodiment, a compound of formula I is described wherein Ar¹ is naphthyl or quinolinyl, each of which is optionally substituted.

In another embodiment, a compound of formula I is described wherein X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, aryl, arylalkyl and heteroarylalkyl, each of which is optionally substituted; or R³ and R⁴ are taken together with the attached carbon to form a cycloalkylene;

In another embodiment, a compound of formula I is described wherein X² is a bond.

In another embodiment, a compound of formula I is described wherein R¹ and X² are taken together with the attached nitrogen to form an optionally substituted heterocycle, and the heterocycle is selected from the group consisting of pyrrolidine, piperidine, piperazine, and homopiperazine, each of which is optionally substituted.

In another embodiment, a compound of formula I is described wherein R¹ and X² are taken together with the attached nitrogen to form an optionally substituted heterocycle, and the heterocycle is selected from the group consisting of pyrrolidine, piperidine, piperazine, and homopiperazine, each of which is optionally substituted

In another embodiment, X² is a bond; and X³ is —C(O)R⁵ , —C(O)OR⁵—C(O)NR⁶R⁵, SO₂NR⁶R⁵, or SO₂R⁵ wherein R⁵ is aryl, heteroaryl, arylalkyl, or heteroarylalkyl, each of which is optionally substituted; and R⁶ are each independently selected from the group consisting of hydrogen, alkyl, arylalkyl, heteroarylalkyl, hydroxyl, alkoxyl and a pro-drug moiety, each of which is optionally substituted.

In another embodiment, a compound of formula I is described wherein X² is a bond; and X³ is aroyl. In another embodiment, a compound of formula I is described wherein X² is a bond; and X³ is aroyl, where the aryl is phenyl, naphthyl, pyridinyl, pyridazinyl, pyrimidinyl, pyrazinyl, thienyl, quinolinyl or quinazolinyl.

In another embodiment, a compound of formula I is described wherein X² is a bond; and X³ is optionally substituted benzoyl. In another embodiment, X² is a bond; and X³ is R^{a}-substituted benzoyl, wherein R^{a} represents 1-4 substituents each of which is independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH₂, alkylamino, dialkylamino, alkylalkylamino, acylamino, urea, carbamate, and the like, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof.

In one variation, R^{a} represents 1-4 substituents each of which is independently selected from the group consisting of hydrogen, halo, hydroxy, optionally substituted alkyl, optionally substituted alkenyl, optionally substituted alkoxy, cyano, acyl, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof.

In another embodiment, a compound of formula II

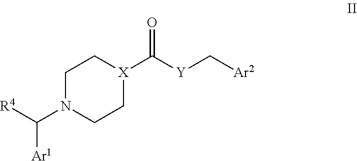

or a pharmaceutically acceptable salt thereof, is described wherein

Ar¹ is aryl or heteroaryl, each of which is optionally substituted;

Ar² is aryl or heteroaryl, each of which is optionally substituted;

R⁴ is hydrogen, alkyl, alkoxyl, arylalkyl or heteroarylalkyl, each of which is optionally substituted;

Y is N(R^{1A}) or 0; where R^{1A} is hydrogen, alkyl, arylalkyl, heteroarylalkyl, hydroxyl, alkoxyl or a pro-drug moiety, each of which is optionally substituted; and

X is CH or N.

In another embodiment, a compound of formula II is described wherein Y is NH.

In another embodiment, a compound of formula IIA

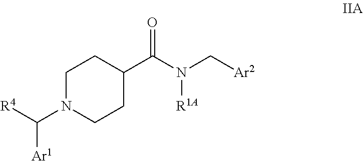

or a pharmaceutically acceptable salt thereof, is described wherein

Ar¹ is aryl or heteroaryl, each of which is optionally substituted;

Ar² is optionally substituted phenyl;

R^{1A} is hydrogen, alkyl, arylalkyl, heteroarylalkyl, hydroxyl, alkoxyl or a pro-drug moiety, each of which is optionally substituted; and

R⁴ is hydrogen, alkyl, alkoxyl, arylalkyl or heteroarylalkyl, each of which is optionally substituted.

In another embodiment of compounds of formulae II and IIA, Ar¹ is naphthyl, quinolinyl, isoquinolinyl, and quinazolinyl, each of which is optionally substituted.

In another embodiment, compounds of formula II and IIA are described wherein Ar¹ is selected from the group consisting of 1-naphthyl, 2-naphthyl, 4-quinolinyl, 4-isoquinolinyl and 4-quinazolinyl, each of which is optionally substituted.

In another embodiment, compounds of formula II and IIA are described wherein Ar¹ is selected from the group consisting of 1-naphthyl, 2-naphthyl, and 4-quinolinyl, each of which is optionally substituted and Y is NH.

In another embodiment of compounds of formulae II and IIA, Ar² is monocyclic aryl or monocyclic heteroaryl, each of which is optionally substituted. In another embodiment of compounds of formulae II and IIA, Ar² is optionally substituted phenyl. In another embodiment of compounds of formulae II and IIA, Ar² is optionally substituted pyrdinyl. In another embodiment of compounds of formulae II and IIA, Ar² is optionally substituted thienyl.

In another embodiment of compounds of formulae II and IIA, Ar² is phenyl substituted with R^{a}, where R^{a} represents 1-4 substituents each of which is independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof.

In one variation, R^{a} represents 1-4 substituents each of which is independently selected from the group consisting of hydrogen, halo, hydroxy, optionally substituted alkyl, optionally substituted alkenyl, optionally substituted alkoxy, cyano, acyl, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof.

In another embodiment, a compound of formula III

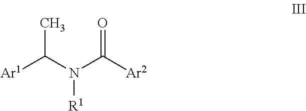

or a pharmaceutically acceptable salt thereof, is described wherein

Ar¹ is aryl or heteroaryl, each of which is optionally substituted;

Ar² is aryl or heteroaryl, each of which is optionally substituted; and

R¹ is hydrogen, alkyl, arylalkyl, heteroarylalkyl, hydroxyl, alkoxyl or a pro-drug moiety, each of which is optionally substituted.

In another embodiment, a compound of formula III is described wherein Ar¹ is selected from naphthyl, quinolinyl, isoquinolinyl, and quinazolinyl, each of which is optionally substituted; and

In another embodiment, a compound of formula III is described wherein Ar² is optionally substituted phenyl. In one variation, Ar² is R^{a}-substituted phenyl, wherein R^{a} represents 1-4 substituents each of which is independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof.

In another variation, R^{a} represents 1-4 substituents each of which is independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof.

In another embodiment, a compound of any of formulae I, II, IIA, or III is described wherein Ar¹ is optionally substituted bicyclic heteroaryl.

In another embodiment, a compound of any of formulae I, II, IIA, or III is described wherein Ar¹ is naphthyl, quinolinyl, or quinazolinyl.

In another embodiment, a compound of any of formulae I, II, IIA, or III is described wherein Ar¹ is naphthyl or quinolinyl.

In another embodiment, a compound of any of formulae I, II, IIA, or III is described wherein Ar¹ is optionally substituted 1-naphthyl. In another embodiment, a compound of any of formulae I, II, IIA, or III is described wherein Ar¹ is optionally substituted 2-naphthyl.

In another embodiment, a compound of any of formulae I, II, IIA, or III is described wherein Ar¹ is optionally substituted 2-quinolinyl. In another embodiment, a compound of any of formulae I, II, IIA, or III is described wherein Ar¹ is optionally substituted 3-quinolinyl. In another embodiment, a compound of any of formulae I, II, IIA, or III is described wherein Ar¹ is optionally substituted 4-quinolinyl. In another embodiment, a compound of any of formulae I, II, IIA, or III is described wherein Ar¹ is optionally substituted 5-quinolinyl. In another embodiment, a compound of any of formulae I, II, IIA, or III is described wherein Ar¹ is optionally substituted 6-quinolinyl. In another embodiment, a compound of any of formulae I, II, IIA, or III is described wherein Arⁱ is optionally substituted 7-quinolinyl. In another embodiment, a compound of any of formulae I, II, IIA, or III is described wherein Ar¹ is optionally substituted 8-quinolinyl.

In another embodiment, a compound of any of formulae I, II, IIA, or III is described wherein Ar¹ is optionally substituted 1-isoquinolinyl. In another embodiment, a compound of any of formulae I, II, IIA, or III is described wherein Ar¹ is optionally substituted 3-isoquinolinyl. In another embodiment, a compound of any of formulae I, II, IIA, or III is described wherein Ar¹ is optionally substituted 4-isoquinolinyl. In another embodiment, a compound of any of formulae I, II, IIA, or III is described wherein Ar¹ is optionally substituted 5-isoquinolinyl. In another embodiment, a compound of any of formulae I, II, IIA, or III is described wherein Ar¹ is optionally substituted 6-isoquinolinyl. In another embodiment, a compound of any of formulae I, II, IIA, or III is described wherein Ar¹ is optionally substituted 7-isoquinolinyl. In another embodiment, a compound of any of formulae I, II, IIA, or III is described wherein Ar¹ is optionally substituted 8-isoquinolinyl.

In another embodiment, a compound of any of formulae I, II, IIA, or III is described wherein Ar¹ is optionally substituted 2-quinazolinyl. In another embodiment, a compound of any of formulae I, II, IIA, or III is described wherein Ar¹ is optionally substituted 4-quinazolinyl. In another embodiment, a compound of any of formulae I, II, IIA, or III is described wherein Ar¹ is optionally substituted 5-quinazolinyl. In another embodiment, a compound of any of formulae I, II, IIA, or III is described wherein Ar¹ is optionally substituted 6-quinazolinyl. In another embodiment, a compound of any of formulae I, II, IIA, or III is described wherein Ar¹ is optionally substituted 7-quinazolinyl. In another embodiment, a compound of any of formulae I, II, IIA, or III is described wherein Ar¹ is optionally substituted 8-quinazolinyl.

In another embodiment, a compound of any of formulae I, II, IIA, or III is described wherein Ar² is optionally substituted monocyclic heteroaryl.

In another embodiment of any of compounds or embodiments of any of formulae I, II, IIA, III, IV, or V, R¹ is hydrogen or a pro-drug moiety.

In another embodiment of any of compounds or embodiments of any of formulae I, II, IIA, III, IV, or V, neither of R³ or R⁴ is H. In another embodiment of any of compounds or embodiments of any of formulae I, II, IIA, III, IV, or V, R³ is H. In another embodiment of any of compounds or embodiments of any of formulae I, II, IIA, III, IV, or V, both R³ and R⁴ are independently selected optionally substituted alkyl. In another embodiment of any of compounds or embodiments of any of formulae I, II, IIA, III, IV, or V, both R³ and R⁴ are methyl. In another embodiment of any of compounds or embodiments of any of formulae I, II, IIA, III, IV, or V, R³ is hydrogen and R⁴ is optionally substituted alkyl. In another embodiment of any of compounds or embodiments of any of formulae I, II, IIA, III, IV, or V, R³ is hydrogen and R⁴ are methyl.

In another embodiment of any of compounds or embodiments of any of formulae I, II, IIA, III, IV, or V, the chirality of the carbon bearing R³ and R⁴ has the following absolute configuration

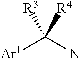

and R⁴ is alkyl, and R³ is hydrogen, alkyl, or alkoxy. In one variation, R³ is hydrogen. In another variation, R⁴ is methyl. In another variation, R³ is hydrogen and R⁴ is methyl.

In another embodiment, a compound of formula IV

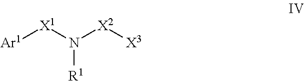

or a pharmaceutically acceptable salt thereof, is described wherein

Ar¹ is 1-napthyl, quinolinyl, isoquinolinyl, or quinazolinyl, each of which is optionally substituted;

X¹ is NR² or CR³R⁴, wherein R² is selected from the group consisting of hydrogen, alkyl, aryl, arylalkyl, heteroaryl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, arylalkyl and heteroarylalkyl, each of which is optionally substituted; or R³ and R⁴ are taken together with the attached carbon to form a cycloalkylene;

R¹ is hydrogen, alkyl, arylalkyl, heteroarylalkyl, hydroxyl, alkoxyl or a pro-drug moiety, each of which is optionally substituted; and X² is selected from the group consisting of a bond, alkylene and heteroalkylene, or R¹ and X² are taken together with the attached nitrogen to form an optionally substituted heterocycle; and

X³ is an acyl group, a carboxylate group, or a derivative thereof, a sulfonate group, or a sulfonamide group.

providing that when X¹ is CR³R⁴, the absolute stereochemistry is (R); and providing that the compound does not have the formula:

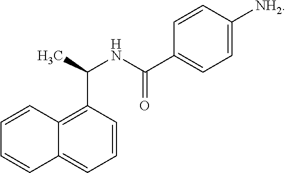

In another embodiment, a compound of formula IV is described wherein X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, aryl, arylalkyl and heteroarylalkyl, each of which is optionally substituted; or R³ and R⁴ are taken together with the attached carbon to form a cycloalkylene;

In another embodiment, a compound of formula IV is described wherein when one of R^{a} is NH₂, then at least one other of R^{a} is other than hydrogen.

In another embodiment, a compound of formula IV is described wherein R^{a} is not NH₂.

In another embodiment, a compound of formula IV is described wherein Ar¹ is 2-quinolinyl, 3-quinolinyl, or 4-quinolinyl.

In another embodiment, a compound of formula V

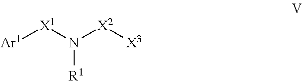

or a pharmaceutically acceptable salt thereof, is described wherein

Ar¹ is optionally substituted 2-napthyl;

X¹ is NR² or CR³R⁴, wherein R² is selected from the group consisting of hydrogen, alkyl, aryl, arylalkyl, heteroaryl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, arylalkyl and heteroarylalkyl, each of which is optionally substituted; or R³ and R⁴ are taken together with the attached carbon to form a cycloalkylene;

R¹ is hydrogen, alkyl, arylalkyl, heteroarylalkyl, hydroxyl, alkoxyl or a pro-drug moiety, each of which is optionally substituted;

X² is selected from the group consisting of a bond, alkylene and heteroalkylene, or

R¹ and X² are taken together with the attached nitrogen to form an optionally substituted heterocycle; and

X³ is an acyl group, a carboxylate group, or a derivative thereof, a sulfonate group, or a sulfonamide group.

providing that when X¹ is CR³R⁴, the absolute stereochemistry is (R); and providing that when X¹ CH(CH₃), R¹ is hydrogen, X² is a bond, and X³ is optionally substituted benzoyl, then X³ includes at least one hydrogen containing hydrogen-bonding group. Illustrative hydrogen containing hydrogen-bonding groups include, but are not limited to, OH, NH₂, NHMe, NHAc, alkylene-NH₂, such as CH₂NH₂ CH₂NHMe, alkylene-OH, such as CH₂OH, and the like.

In another embodiment, a compound of formula V is described wherein X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, aryl, arylalkyl and heteroarylalkyl, each of which is optionally substituted; or R³ and R⁴ are taken together with the attached carbon to form a cycloalkylene;

In another embodiment a compound of formula VI is described

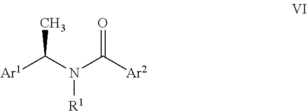

or a pharmaceutically acceptable salt thereof, is described wherein

Ar¹ is 1-napthyl, quinolinyl, isoquinolinyl, and quinazolinyl, each of which is optionally substituted;

R¹ is hydrogen, alkyl, arylalkyl, heteroarylalkyl, hydroxyl, alkoxyl or a pro-drug moiety, each of which is optionally substituted; and X² is selected from the group consisting of a bond, alkylene and heteroalkylene, or R¹ and X² are taken together with the attached nitrogen to form an optionally substituted heterocycle; and

Ar² is optionally substituted phenyl;

providing that the compound does not have the formula:

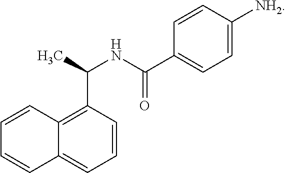

In another embodiment, compounds of any one of formulae IV, V, or VI are described wherein X² is a bond.

In another embodiment, compounds of any one of formulae IV, V, or VI are described wherein R¹ and X² are taken together with the attached nitrogen to form an optionally substituted heterocycle, and the heterocycle is selected from the group consisting of pyrrolidine, piperidine, piperazine, and homopiperazine, each of which is optionally substituted.

In another embodiment, compounds of any one of formulae IV, V, or VI are described wherein R¹ and X² are taken together with the attached nitrogen to form an optionally substituted heterocycle, and the heterocycle is selected from the group consisting of pyrrolidine, piperidine, piperazine, and homopiperazine, each of which is optionally substituted

In another embodiment, compounds of any one of formulae IV, V, or VI are described wherein X² is a bond; and X³ is —C(O)R⁵, —C(O)OR⁵—C(O)NR⁶R⁵, SO₂NR⁶R⁵, of SO₂R⁵ wherein R⁵ is aryl, heteroaryl, arylalkyl, or heteroarylalkyl, each of which is optionally substituted; and R⁶ are each independently selected from the group consisting of hydrogen, alkyl, arylalkyl, heteroarylalkyl, hydroxyl, alkoxyl and a pro-drug moiety, each of which is optionally substituted.

In another embodiment, compounds of any one of formulae IV, V, or VI are described wherein X² is a bond; and X³ is aroyl. In another embodiment, a compound of formula IV is described wherein X² is a bond; and X³ is aroyl, where the aryl is phenyl, naphthyl, pyridinyl, pyridazinyl, pyrimidinyl, pyrazinyl, thienyl, quinolinyl or quinazolinyl.

In another embodiment, compounds of any one of formulae IV, V, or VI are described wherein X² is a bond; and X³ is optionally substituted benzoyl. In another embodiment, X² is a bond; and X³ is R^{a}-substituted benzoyl, wherein R^{a} represents 1-4 substituents each of which is independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of halo, hydroxy, optionally substituted alkyl, optionally substituted alkenyl, optionally substituted heteroalkyl, such as alkoxyalkyl, aminoalkyl, it being understood that amino includes NH₂, alkylamino, dialkylamino, alkylalkylamino, and the like, and when optionally substituted includes acylamino, and the like, optionally substituted alkoxy, cyano, acyl, optionally substituted amino, such as NH2, alkylamino, dialkylamino, alkylalkylamino, acylamino, urea, carbamate, and the like, nitro, optionally substituted alkylthio, optionally substituted alkylsulfonyl, and carboxylic acid and derivatives thereof.

In one variation, R^{a} represents 1-4 substituents each of which is independently selected from the group consisting of hydrogen, halo, hydroxy, optionally substituted alkyl, optionally substituted alkenyl, optionally substituted alkoxy, cyano, acyl, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof.

In another embodiment, compounds of formulae IV-VI are described wherein the heterocycle is selected from the group consisting of pyrrolidine, piperidine, piperazine, and homopiperazine, each of which is optionally substituted.

In another embodiment compounds of formulae IV-VI are described wherein X³ is benzoyl, or substituted benzoyl. In another embodiment, X³ is benzoyl substituted with between 1 and 4 substituents each of which is independently selected from the group consisting of halo, hydroxy, optionally substituted alkyl, optionally substituted alkenyl, optionally substituted alkoxy, cyano, acyl, nitro, optionally substituted alkylthio, optionally substituted alkylsulfonyl, and carboxylic acid and derivatives thereof; or R^{a} represents 2-4 substituents where 2 of said substituents are adjacent substituents and are taken together with the attached carbons to form an optionally substituted heterocycle, and where the remaining substituents, in cases where R^{a} represents 3-4 substituents, are each independently selected from the group consisting of hydrogen, halo, hydroxy, optionally substituted alkyl, optionally substituted alkenyl, optionally substituted alkoxy, cyano, nitro, optionally substituted alkylthio, optionally substituted alkylsulfonyl, and carboxylic acid and derivatives thereof.

In another embodiment of any of compounds or embodiments of any of formulae I, II, IIA, III, IV, or V, neither of R³ or R⁴ is H.

In another embodiment of any of compounds or embodiments of any of formulae I, II, IIA, III, IV, or V, the chirality of the carbon bearing R³ and R⁴ has the following absolute configuration

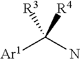

when R⁴ has a higher Cahn-Ingold-Prelog priority than R³. For example, in one variation, R³ is H and R⁴ is alkyl, such as methyl. In that variation, the absolute configuration of the chiral carbon is (R). In another variation, R³ is alkyl, and R⁴ is alkoxyalkyl. In that variation, the absolute configuration of the chiral carbon is (R).

In another embodiment of any of compounds or embodiments of any of formulae I, II, IIA, III, IV, or V, the chirality of the carbon bearing R³ and R⁴ has the following absolute configuration

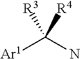

and R⁴ is alkyl, and R³ is hydrogen, alkyl, or alkoxy.

In another embodiment of the method the compounds described in Table 1 are described.

TABLE 1

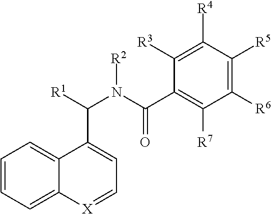

| Compound | X | R₁ | R₂ | R₃ | R₄ | R₅ | R₆ | R₇ |
|---|---|---|---|---|---|---|---|---|
| 1-1 | CH | S—Me | H | Me | H | H | H | H |
| 1-2 | CH | S—Me | H | H | Me | H | H | H |
| 1-3 | CH | S—Me | H | H | H | Me | H | H |
| 5h | CH | R—Me | H | Me | H | H | H | H |
| 1-5 | CH | R—Me | H | H | Me | H | H | H |
| 1-6 | CH | R—Me | H | H | H | Me | H | H |
| 1-7 | CH | R—Me | H | Me | H | H | H | Me |
| 1-8 | CH | R—Me | H | OH | H | H | NH₂ | H |
| 22 | CH | R—Me | H | H | H | NHBoc | H | H |
| 23 | CH | R—Me | H | H | H | NH₂ | H | H |
| 5i | CH | R—Me | H | Me | H | H | NO₂ | H |
| 24 | CH | R—Me | H | Me | H | H | NH₂ | H |
| 25 | CH | R—Me | H | Me | H | H | NHac | H |
| 21 | CH | R—Me | Me | Me | H | H | H | H |
| 1-15 | CH | Et(rac) | H | Me | H | H | H | H |
| 47 | CH | R—Me | H | Me | H | H | CH₂NHBoc | H |
| 2 | CH | R—Me | H | Me | H | H | CH₂NH₂ | H |
| 1-19 | CH | R—Me | H | Me | H | H | CH₂N(CH₃)Boc | H |
| 49 | CH | R—Me | H | Me | H | H | CH₂NHCH₃ | H |
| 28 | CH | di-Me | H | Me | H | H | NO₂ | H |
| 29 | CH | di-Me | H | Me | H | H | NH₂ | H |
| 50 | N | Me(rac) | H | Me | H | H | NH₂ | H |
| 51 | N | Me(rac) | H | Me | H | NO₂ | H | H |
| 52 | N | Me(rac) | H | Me | H | NH₂ | H | H |
| 53 | N | Me(rac) | H | Me | H | H | CN | H |
| 54 | N | Me(rac) | H | Me | H | H | CH₂OH | H |

In one variation of the various embodiment of compounds described herein, the invention does not include compound 23 or its enantiomer.

In another embodiment of the method the compounds described in Table 2 are described.

TABLE 2

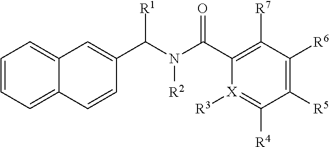

| Compound | R₁ | R₂ | R₃ | R₄ | R₅ | R₆ | R₇ | X |
|---|---|---|---|---|---|---|---|---|
| 1a | S—Me | H | Me | H | H | H | H | C |
| 2-2 | S—Me | H | H | Me | H | H | H | C |
| 2-3 | S—Me | H | H | H | Me | H | H | C |
| 2-4 | S—Me | H | OMe | H | H | H | H | C |
| 2-5 | S—Me | H | H | OMe | H | H | H | C |
| 2-6 | S—Me | H | H | H | OMe | H | H | C |
| 2-7 | S—Me | H | None | H | H | H | H | N |
| 2-8 | S—Me | H | O | H | H | H | H | N |
| 1b | R—Me | H | Me | H | H | H | H | C |
| 5a | R—Me | H | H | Me | H | H | H | C |
| 5b | R—Me | H | H | H | Me | H | H | C |
| 5c | R—Me | H | OMe | H | H | H | H | C |
| 5d | R—Me | H | H | OMe | H | H | H | C |
| 5e | R—Me | H | H | H | OMe | H | H | C |
| 2-15 | R—Me | H | None | H | H | H | H | N |
| 2-16 | R—Me | H | O | H | H | H | H | N |
| 2-17 | R—Me | H | Et | H | H | H | H | C |
| 2-18 | R—Me | H | Me | H | H | —(CH₂)₄—R₇ | —(CH₂)₄—R ₆ | C |
| 2-19 | R—Me | H | Ph | H | H | H | H | C |
| 5f | R—Me | H | Me | H | H | H | Me | C |
| 2-21 | R—Me | H | Me | H | H | H | Br | C |
| 5g | R—Me | H | OH | H | H | H | H | C |
| 2-23 | R—Me | H | OAc | H | H | H | H | C |
| 2-24 | R—Me | H | Me (mixture) | H₂ | H₂ | H₂ | H₂ | CH |
| 2-25a | R—Me | H | Cl | H | H | H | H | C |
| 2-25b | S—Me | H | Cl | H | H | H | H | C |
| 2-26 | R—Me | H | OH | H | H | NHBoc | H | C |
| 2-27 | R—Me | H | OH | H | H | NH₂ | H | C |
| 2-28 | R—Me | H | NHBoc | H | H | H | H | C |
| 2-29 | R—Me | H | NH₂ | H | H | H | H | C |
| 2-30 | R—Me | Me | Me | H | H | H | H | C |
| 8 | R—Me | H | H | H | NHBoc | H | H | C |
| 9 | R—Me | H | H | H | NH₂ | H | H | C |
| 2-33 | R—Me | H | Me | H | H | NO₂ | H | C |
| 2-34 | R—Me | H | Me | H | H | NH₂ | H | C |
| 14 | Et(rac) | H | Me | H | H | H | H | C |
| 17 | Ph(rac) | H | Me | H | H | H | H | C |

In one variation of the various embodiments of compounds described herein, the invention does not include compounds 2-1, 2-5, 2-6, 2-7, 1b, 5c, 5e, 2-15, 2-25a, or 2-25b, or their racemic forms; or the racemic form of 2-27.

In another embodiment of the method the compounds described in Table 3 are described.

TABLE 3

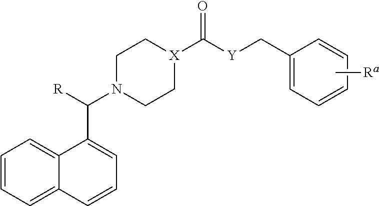

| Compound | R | X | Y | R^{a} |
|---|---|---|---|---|
| 68 | S—Me | CH | NH | m,p-dioxolane |
| 69 | R—Me | CH | NH | m,p-dioxolane |
| 64 | R—Me | CH | NH | m-OMe |
| 63 | R—Me | CH | NH | p-OMe |
| 67 | R—Me | CH | NH | o-OMe |
| 3-6 | H | CH | NH | m,p-dioxolane |
| 65 | Me(rac) | N | O | p-OMe |
| 66 | Me(rac) | N | NH | p-OMe |
| 3-9 | di-Me | CH | NH | m,p-dioxolane |

In another embodiment of the method the compounds described in Table 4 are described.

TABLE 4

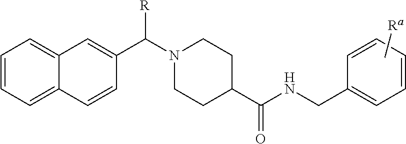

| Compound | R | R^{a} |
|---|---|---|
| 61 | S—Me | m-OMe |
| 62 | R—Me | m-OMe |
| 4-3 | R—Me | o-OMe |
| 4-4 | H | m,p-dioxolane |

In another embodiment, a pharmaceutical composition or pharmaceutical formulation in unit dosage form is described. In one aspect, the composition or formulation includes an effective amount of one or more compounds described herein, including any one or any combination of compounds of formulae I, II, IIA, III, IV, V, and/or VI, for treating a respiratory disease or illness. It is to be understood that combinations and/or mixtures of the compounds described herein may be included in the composition or formulation. In another embodiment, the composition or formulation includes an effective amount for treating SARS in a patient in need of relief.

All of the compounds described herein can be prepared by conventional routes such as by the procedures described in the general methods presented herein or by the specific methods described in the Methods section, or by similar methods thereto. The present invention also encompasses any one or more of these processes for preparing the compounds described herein, in addition to any novel intermediates used therein.

Illustratively, in another embodiment, processes for preparing the compounds are described in the following illustrative examples and schemes.

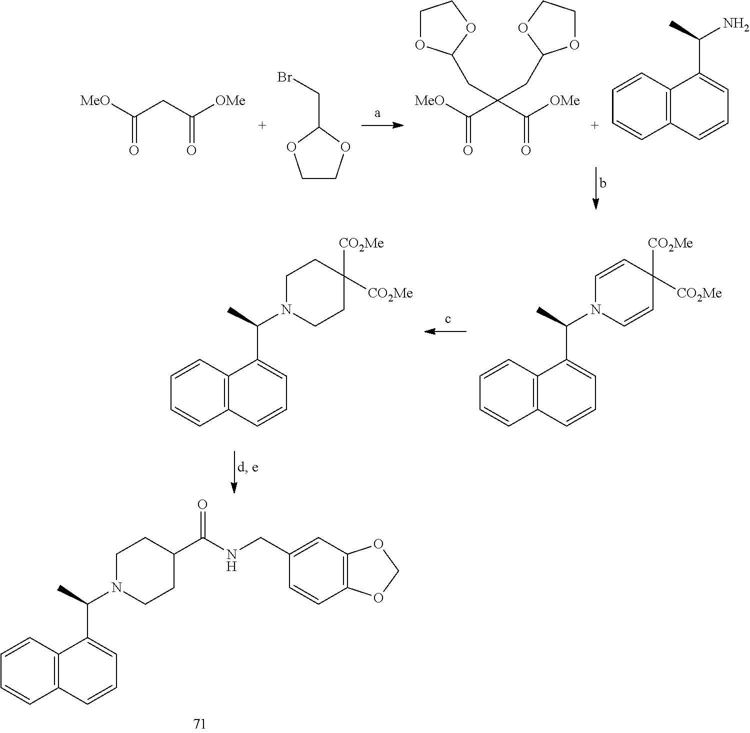

(a) KOt-Bu, DMSO, rt, 48 h; (b) 10% HCl aq., THF, rt, 18 h; (c) H2, Pd—C, EtOAc, rt, 15 h; (d) 0.1M NaOH aq., MeOH, reflux, 3 h; (e) 3,4-methylenedioxy-benzylamine, EDCI, HOBT, DIPEA, CH₂Cl₂, rt, 16 h. In other variations, other substituted benzylamines can be used in the amide forming step.

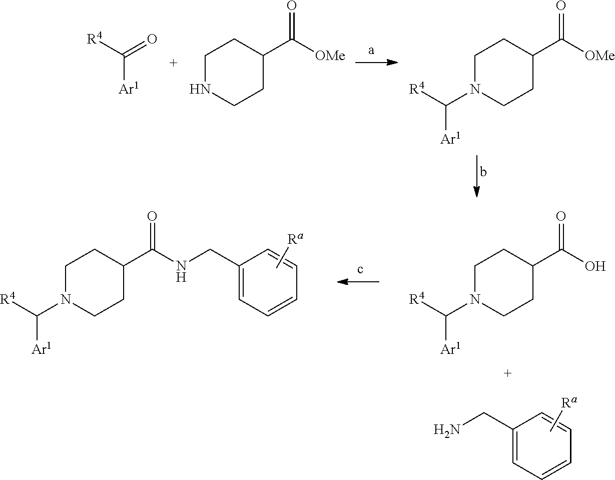

Reagent and conditions: (a) HOAc, NaBH₃CN, MeOH, 24 h, 23° C.; ; (b) LiOH.H₂O, THF/H₂O (5:1), 23° C., 1.5 h; (c) EDCI, HOBT, DIPEA, DMF, 23° C., 16 h.

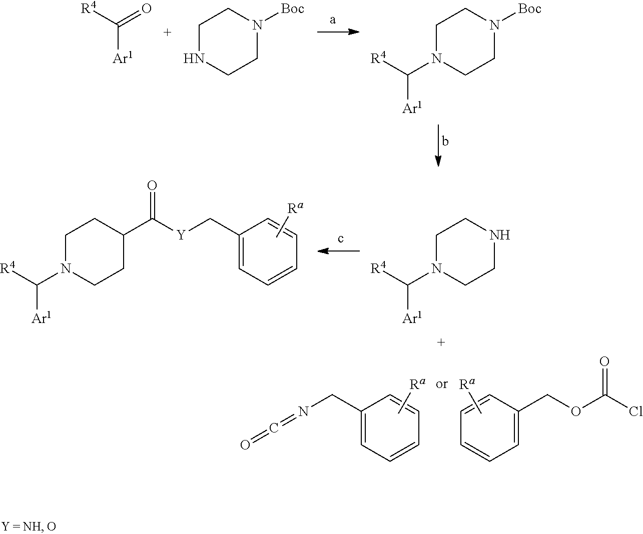

(a) HOAc, NaBH₃CN, MeOH, 24 h, 23° C.; (b) TFA, CH₂Cl₂, 23° C., 2 h; (c) CH₂Cl₂, 0-23° C., 1-24 h.

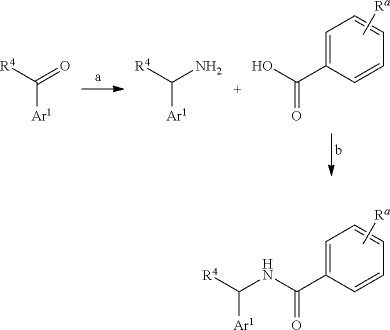

(a) NH₄OAc, NaBH₃CN, MeOH, 23° C., 24 h; (b) EDCI, HOBT, DIPEA, DMF, 23° C., 16 h.

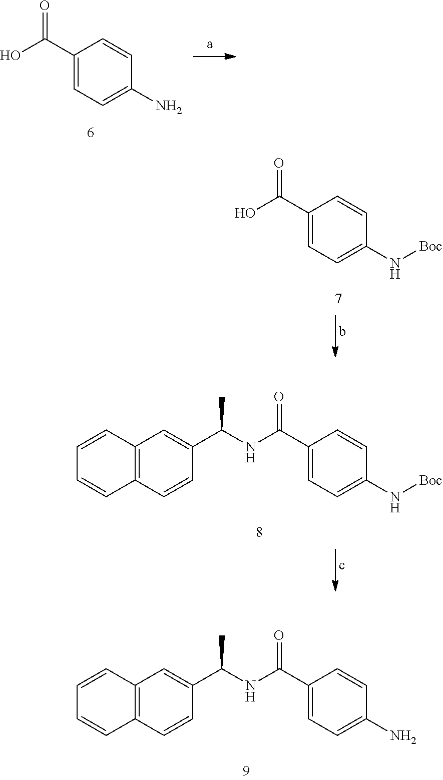

Reagents and conditions: (a) Boc₂O, Et₃N, dioxane/H₂O (2:1), 23° C., 48 h; (b) (R)-(+)-1-(2-naphthyl)ethylamine, EDCI, HOBT, DIPEA, CH₂Cl₂, 23° C., 16 h; (c) TFA, CH₂Cl₂, 23° C., 2 h.

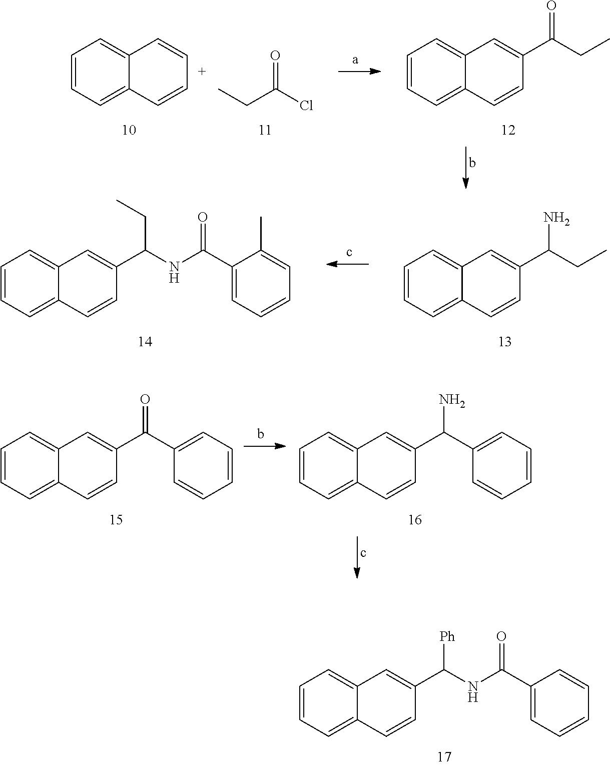

Reagent and conditions: (a) A1Cl₃, 1,2-dichloroethane, 35° C., 4 h; (b) NH₄OAc, NaBH₃CN, MeOH, 23° C., 24 h; (c) o-toluic acid, EDCI, HOBT, DIPEA, DMF, 23° C., 16 h.

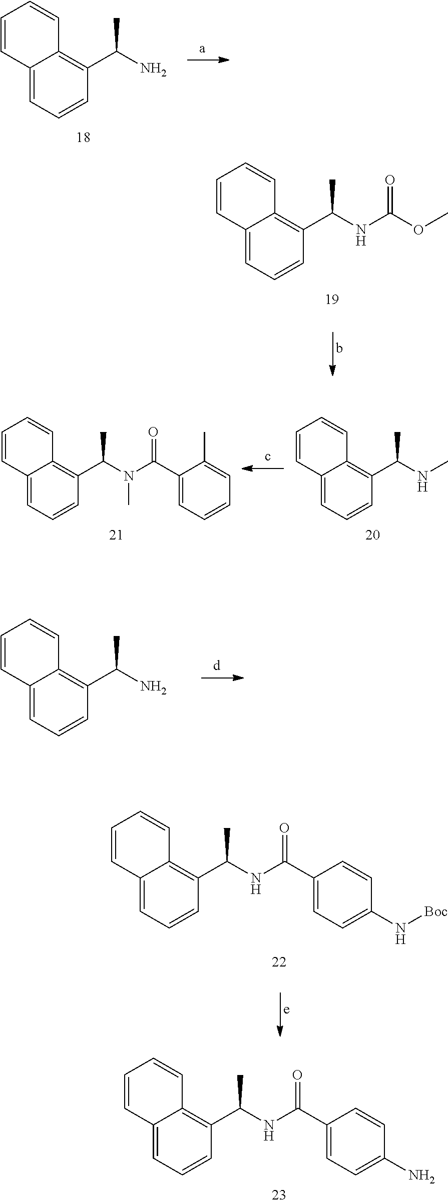

Reagents and conditions: (a) ClCO₂Me,K₂CO₃, dioxane/H₂O(1:1), 0° C., 1 h; (b) LiAlH₄, THF, reflux, 1 h; (c) o-toluic acid, EDCI, HOBT, DIPEA, DMF, 23° C., 16 h; (d) 7, EDCI, HOBT, DIPEA, DMF, 23° C., 16 h; (e) TFA, CH₂Cl₂, 23° C., 2 h.

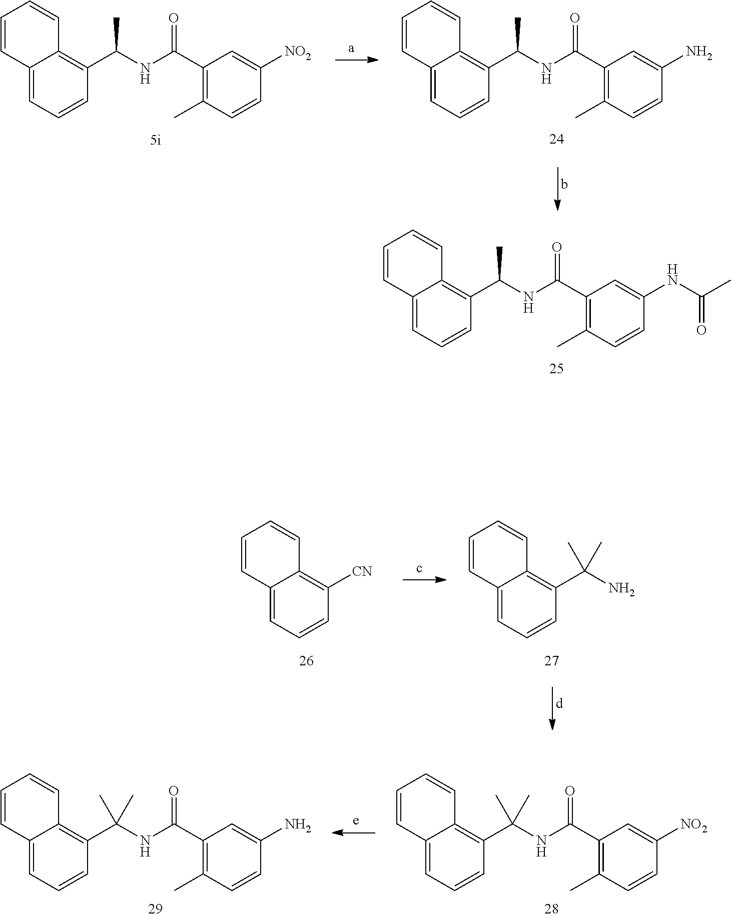

Reagents and conditions: (a) H₂, Pd—C, EtOAc/MeOH(1:1), 23° C., 15 h; (b) Ac₂O, Et₃N,CH₂Cl₂, 23° C., 18 h; (c) MeLi, CeCl₃, THF, 23° C., 2 h; nitrobenzoic acid, EDCI, HOBT, DIPEA, CH₂Cl₂, 23° C., 16 h; (e) H₂, Pd—C, EtOAc/MeOH (1:1), 23° C., 15 h.

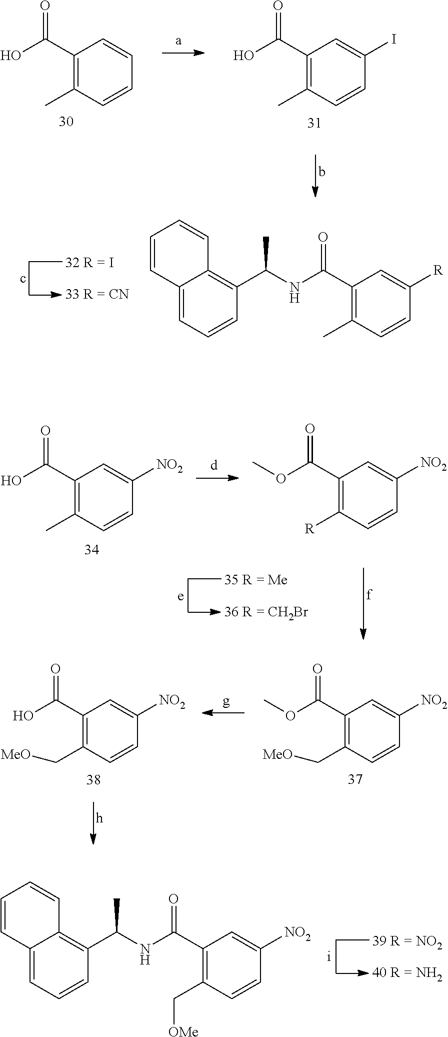

Reagents and conditions: (a) KI, NaIO₄, conc H₂SO₄, 25-30° C., 2 h; (b) (R)-(+)-1-(1-naphthyl)ethylamine 18, EDCI, HOBT, DIPEA, DMF/CH₂Cl₂ (1:1), 23° C., 48 h; (c) CuCN, KCN, DMF, 130° C., 16 h; (d) SOCl₂, MeOH, reflux, 4 h; (e) NBS, Bz₂O₂, CCl₄, reflux, 24 h; (f) NaH, NaOMe, MeOH, 50° C., 4 h; (g) LiOH.H₂O, THF/H₂O (5:1), 23° C., 1.5 h; (h) (R)-(+)-1-(1-naphthyl)ethylamine 18, EDCI, HOBT, DIPEA, DMF/CH₂Cl₂ (1:1), 23° C., 16 h; (i) H₂, Pd—C, EtOAc, 23° C., 10 h.

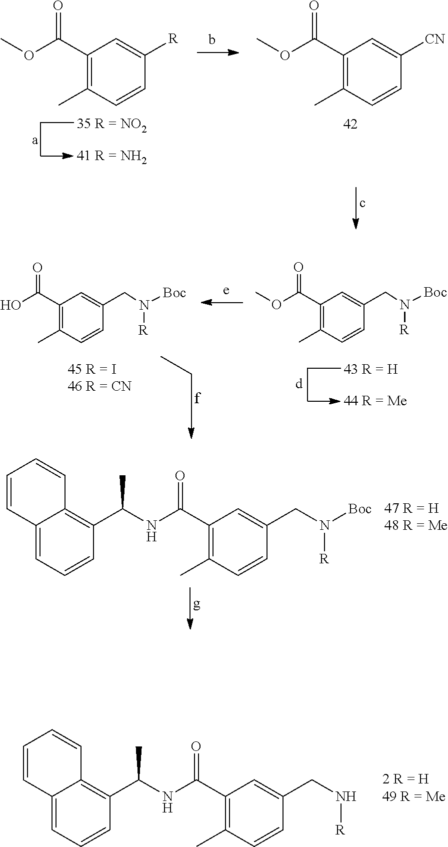

Reagents and conditions: (a) H₂, Pd—C, EtOAc, 23° C., 16 h; (b) NaNO₂, conc HCl, CuCN, NaCN, H₂O, 23° C., 3 h; (c) Boc₂O, NiCl₂.6H₂O, NaBH4, MeOH, 23° C., 2 H; (d) MeI, KHMDS, THF, 23° C., 16 h; (e) LiOH.H₂O, THF/H₂O(9:1), 23° C., 16 h; (f) (R)-(+)-1-(1-naphthyl)ethylamine 18, EDCI, HOBT, DIPEA, CH₂Cl₂, 23° C., 16 h; (g) TFA, CH₂Cl₂, 23° C., 2 h.

It is to be understood that the foregoing processes may be adapted using conventional techniques and the appropriate selection of the corresponding starting materials to prepare the compounds described herein.

In this and other embodiments described herein, it is understood that the compounds may be neutral or may be one or more pharmaceutically acceptable salts, crystalline forms, non crystalline forms, hydrates, or solvates, or a combination of the foregoing. Accordingly, all references to the compounds described herein may refer to the neutral molecule, and/or those additional forms thereof collectively and individually from the context. Pharmaceutically acceptable salts of the compounds described herein include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

The compounds described herein may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other the compounds described herein or in combination with one or more other drugs (or as any combination thereof). Generally, they will be administered as a formulation in association with one or more pharmaceutically acceptable excipients. The term ‘excipient’ is used herein to describe any ingredient other than the compounds described herein. The choice of excipient will to a large extent depend on factors such as the particular mode of administration, the effect of the excipient on solubility and stability, and the nature of the dosage form.

Pharmaceutical compositions suitable for the delivery of the compounds described herein and methods for their preparation will be readily apparent to those skilled in the art. Such compositions and methods for their preparation may be found, for example, in Remington: The Science and Practice of Pharmacy, (21^{st} ed., 2005).

The compounds described herein may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

The compounds described herein may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the compounds described herein may make up from 1 weight % to 80 weight % of the dosage form, more typically from 5 weight % to 60 weight % of the dosage form. In addition to the compounds described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (as, for example, the monohydrate, spray-dried monohydrate or anhydrous form), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% of one or more of the compounds described herein, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

Solid formulations for oral administration may be formulated to be immediate and/or modified release. Modified release formulations include delayed, sustained, pulsed, controlled, targeted and programmed release formulations.

The compounds described herein may also be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular and subcutaneous delivery. Suitable means for parenteral administration include needle (including microneedle) injectors, needle-free injectors, and infusion techniques.

Parenteral formulations are typically aqueous solutions which may contain excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the compounds described herein used in the preparation of a parenteral formulation may be increased by the use of appropriate formulation techniques, such as the incorporation of solubility-enhancing agents.

Formulations for parenteral administration may be formulated to be immediate and/or modified release. Modified release formulations include delayed, sustained, pulsed, controlled, targeted and programmed release formulations. Thus, the compounds described herein may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebuliser contains a solution or suspension of one or more of the compounds described herein comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of one or more of the compounds described herein per actuation and the actuation volume may vary from 1 μl to 100 μl. A typical formulation may comprise one or more of the compounds described herein, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the invention are typically arranged to administer a metered dose or “puff”. The overall daily dose will be administered in a single dose or, more usually, as divided doses throughout the day.

The compounds described herein may contain one or more chiral centers, or may otherwise be capable of existing as multiple stereoisomers. Accordingly, it is to be understood that the present invention includes pure stereoisomers as well as mixtures of stereoisomers, such as enantiomers, diastereomers, and enantiomerically or diastereomerically enriched mixtures. The compounds described herein may be capable of existing as geometric isomers. Accordingly, it is to be understood that the present invention includes pure geometric isomers or mixtures of geometric isomers.

Effective doses of the present compounds depend on many factors, including the indication being treated, the route of administration, co-administration of other therapeutic compositions, and the overall condition of the patient. For oral administration, for example, effective doses of the present compounds herein described are from about 0.01 mg/kg to about 50 mg/kg, from about 0.1 mg/kg to about 50 mg/kg, from 0.5 mg/kg to about 25 mg/kg, from about 0.5 mg/kg to about 10 mg/kg, 0.5 mg/kg to about 5 mg/kg, from about 1 mg/kg to about 10 mg/kg, and the like. Effective parenteral doses can range from about 0.01 to about 50 mg/kg of body weight. In general, treatment regimens utilizing compounds described herein comprise administration of from about 1 mg to about 500 mg of the compounds of this invention per day in multiple doses or in a single dose.

The term “cycloalkyl” as used herein refers to a monovalent chain of carbon atoms, at least a portion of which forms a ring. The term “cycloalkenyl” as used herein refers to a monovalent chain of carbon atoms containing one or more unsaturated bonds, at least a portion of which forms a ring.

The term “heterocycloalkyl” as used herein generally refers to a monovalent chain of carbon atoms and heteroatoms, at least a portion of which forms a ring. The term “heterocycloalkenyl” as used herein refers to a monovalent chain of carbon atoms and heteroatoms containing one or more unsaturated bonds, a portion of which forms a ring, wherein the heteroatoms are selected from nitrogen, oxygen or sulfur.

As used herein, the term “alkylene” is generally refers to a bivalent saturated hydrocarbon group wherein the hydrocarbon group may be a straight-chained or a branched-chain hydrocarbon group. Non-limiting illustrative examples include methylene, 1,2-ethylene, 1-methyl-1,2-ethylene, 1,4-butylene, 2,3-dimethyl-1,4-butylene, 2-methyl-2-ethyl-1,5-pentylene, and the like.

The terms “heteroalkyl” and “heteroalkylene” as used herein includes molecular fragments or radicals comprising monovalent and divalent, respectively, groups that are formed from a linear or branched chain of carbon atoms and heteroatoms, wherein the heteroatoms are selected from nitrogen, oxygen, and sulfur, such as alkoxyalkyl, alkyleneoxyalkyl, aminoalkyl, alkylaminoalkyl, alkyleneaminoalkyl, alkylthioalkyl, alkylenethioalkyl, alkoxyalkylaminoalkyl, alkylaminoalkoxyalkyl, alkyleneoxyalkylaminoalkyl, and the like. It is to be understood that neither heteroalkyl nor heteroalkylene includes oxygen-oxygen fragments. It is also to be understood that neither heteroalkyl nor heteroalkylene includes oxygen-sulfur fragments, unless the sulfur is oxidized as S(O) or S(O)₂.

As used herein, “haloalkyl” is generally taken to mean an alkyl group wherein one or more hydrogen atoms is replaced with a halogen atom, independently selected in each instance from the group consisting of fluorine, chlorine, bromine and iodine. Non-limiting, illustrative examples include, difluoromethly, 2,2,2-trifluoroethyl, 2-chlorobutyl, 2-chloro-2-propyl, trifluoromethyl, bromodifluoromethyl, and the like.

As used herein, the term “optionally substituted” includes a wide variety of groups that replace one or more hydrogens on a carbon, nitrogen, oxygen, or sulfur atom, including monovalent and divalent groups. Illustratively, optional substitution of carbon includes, but is not limited to, halo, hydroxy, alkyl, alkoxy, haloalkyl, haloalkoxy, aryl, arylalkyl, acyl, acyloxy, and the like. In one aspect, optional substitution of aryl carbon includes, but is not limited to, halo, amino, hydroxy, alkyl, alkenyl, alkoxy, arylalkyl, arylalkyloxy, hydroxyalkyl, hydroxyalkenyl, alkylene dioxy, aminoalkyl, where the amino group may also be substituted with one or two alkyl groups, arylalkylgroups, and/or acylgroups, nitro, acyl and derivatives thereof such as oximes, hydrazones, and the like, cyano, alkylsulfonyl, alkylsulfonylamino, and the like. Illustratively, optional substitution of nitrogen, oxygen, and sulfur includes, but is not limited to, alkyl, haloalkyl, aryl, arylalkyl, acyl, and the like, as well as protecting groups, such as alkyl, ether, ester, and acyl protecting groups, and pro-drug groups. Illustrative protecting groups contemplated herein are described in Greene & Wuts “Greene's Protective Groups in Organic Synthesis” 4th Ed., John Wiley & Sons, (NY, 2006). I t is further understood that each of the foregoing optional substituents may themselves be additionally optionally substituted, such as with halo, hydroxy, alkyl, alkoxy, haloalkyl, haloalkoxy, and the like.

As used herein, the term “alkyl” refers to a saturated monovalent chain of carbon atoms, which may be optionally branched, the term “alkenyl” refers to an unsaturated monovalent chain of carbon atoms including at least one double bond, which may be optionally branched, the term “alkylene” refers to a saturated bivalent chain of carbon atoms, which may be optionally branched, and the term “cycloalkylene” refers to a saturated bivalent chain of carbon atoms, which may be optionally branched, a portion of which forms a ring.

As used herein, the term “heterocycle” refers to a chain of carbon and heteroatoms, wherein the heteroatoms are selected from nitrogen, oxygen, and sulfur, at least a portion of which, including at least one heteroatom, form a ring, such as, but not limited to, tetrahydrofuran, aziridine, pyrrolidine, oxazolidine, 3-methoxypyrrolidine, 3-methylpiperazine, and the like.

As used herein, the term “acyl” refers to hydrogen, alkyl, cycloalkyl, alkenyl, cycloalkenyl, heterocyclyl, optionally substituted aryl, optionally substituted arylalkyl, optionally substituted heteroaryl, and optionally substituted heteroarylalkyl attached as a substituent through a carbonyl (C═O) group, such as, but not limited to, formyl, acetyl, pivalolyl, benzoyl, phenacetyl, and the like.

As used herein, the term “aroyl” refers to an optionally substituted aryl or an optionally substituted heteroaryl attached through a carbonyl group.

As used herein, the term “amino” includes the group NH₂, alkylamino, and dialkylamino, where the two alkyl groups in dialkylamino may be the same or different, i.e. alkylalkylamino. Illustratively, amino include methylamino, ethylamino, dimethylamino, methylethylamino, and the like. In addition, it is to be understood that when amino modifies or is modified by another term, such as aminoalkyl, or acylamino, the above variations of the term amino continue to apply. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “optionally substituted amino” includes derivatives pf amino as described herein, such as, but not limited to, acylamino, urea, and carbamate, and the like.

As used herein, the term “prodrug” generally refers to groups that are labile in vivo under predetermined biological conditions, and include, but are not limited to, groups such as (C₃-C₂₀)alkanoyl; halo-(C₃-C₂₀)alkanoyl; (C₃-C₂₀)alkenoyl: (C₄-C₇)cycloalkanoy; (C₃-C₆)-cycloalkyl(C₂-C₁₆)alkanoyl; aroyl which is unsubstituted or substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; aryl(C₂-C₁₆)alkanoyl which is unsubstituted or substituted in the aryl moiety by 1 to 3 substituents selected from the group consisting of halogen, (C₁-C₃)alkyl and (C₁-C₃)alkoxy, each of which is optionally further substituted with 1 to 3 halogen atoms; and hetero-arylalkanoyl having one to three heteroatoms selected from O, S and N in the heteroaryl moiety and 2 to 10 carbon atoms in the alkanoyl moiety and which is unsubstituted or substituted in the heteroary1 moiety by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms.

It is also appreciated that in the foregoing embodiments, certain aspects of the compounds are presented in the alternative, such as selections for any one or more of X, X¹, X², X³, Ar¹, Ar², R^{a}, R¹, R^{1A}, R², R³, R⁴, R⁵, R⁶, and Y. It is therefore to be understood that various alternate embodiments of the invention include individual members of those lists, as well as the various subsets of those lists. Each of those combinations are to be understood to be described herein by way of the lists. Illustrative examples include the method, composition or compound wherein Ar¹ is 1-naphthyl or 4-quinolinyl; R³ is hydrogen; R⁴ is alkyl; and X³ is aroyl; or wherein Ar¹ is 1-naphthyl; X² and R¹ form a piperidine; and X² is a derivative of a carboxylate; or wherein Ar¹ is aryl or heteroaryl; X¹ is the R-isomer of —(CH³)CH—; X² is a bond; and X³ is R^{a}-substituted benzoyl, where R^{a} is (2-Me, 5-NH₂).

Identification of a SARS-CoV PLpro Inhibitor. The numerous functions and requisite roles of PLpro in viral replication and pathogenesis suggest that PLpro may serve as a target for antiviral drugs. Described herein is a sensitive, fluorescence-based high-throughput screen used to identify potential inhibitors of PLpro. This screen is based on previous studies which showed that PLpro is more catalytically active toward ubiquitin-derived substrates relative to polyprotein-based peptide substrates (Barretto N, et al. (2005)). Described herein is the use of a commercially-available peptide substrate representing the 5 C-terminal residues of ubiquitin derivatized with a C-terminal 7-amido-4-methylcoumarin (AMC) fluorogenic reporter group of the following formula

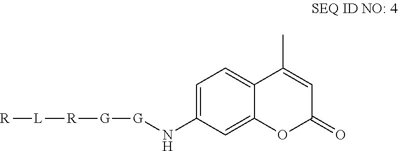

Also described is a pre-screen of 10,000 diverse compounds in the absence of reducing agent to assess the reactivity of PLpro's active site cysteine with electrophiles common to many diverse compound libraries. The vast majority of hits displaying >60% inhibition were determined to be either known electrophiles or to exhibit no inhibitory activity in the presence of reducing agent during follow-up analysis. Although the majority of cysteine protease inhibitors described in the literature act covalently, the inherent electrophilic nature of these compounds often leads to non-specific reactivity with non-targeted nucleophiles, resulting in adverse side effects (Ziebuhr J, et al. (2007)). In the interest of discovering and developing only non-covalent inhibitors against PLpro, 5 mM dithiothreitol (DTT) was incorporated into all subsequent, primary high-throughput screens.

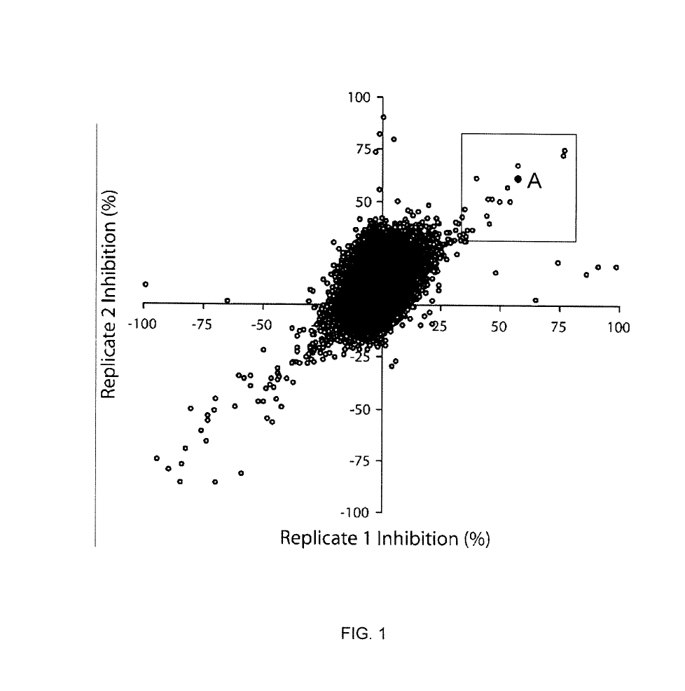

A primary screen of more than 50,000 diverse, lead-like and drug-like compounds was performed in 384-well plates, in duplicate, which resulted in a Z′-factor of 0.8. Only a small number of compounds, 17 total (0.04%), were found to have >35% inhibitory activity toward PLpro (see, FIG. 1). These 17 compounds were subjected to a series of confirmatory and secondary assays to test for interference of AMC fluorescence, dose-dependent inhibition of PLpro, and inhibition of the enzyme in the presence of Triton-X, a test to eliminate promiscuous inhibitors (Feng B Y & Shoichet B K (2006) A Detergent-Based Assay For The Detection Of Promiscuous Inhibitors. Nat Protoc 1(2):550-553). Of the original 17 hits, 9 compounds were found to interfere with the fluorescence of the AMC reporter group, and of the remaining 8 compounds, only two compounds reproducibly inhibited PLpro in a dose-dependent manner, both in the absence and presence of Triton-X. Compound 1, a racemic mixture of 2-methyl-N-[1-(2-naphthyl)ethyl]benzamide (FIG. 1, solid dot), inhibited PLpro with an IC₅₀ value of 20.1±1.1 μM, as shown in the following Table 5 with other compounds described herein.

TABLE 5

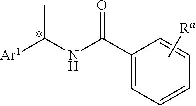

| | | | | IC₅₀ | EC₅₀ |
|---|---|---|---|---|---|
| Compound | isomer* | R^{a} | Ar¹ | (μM) | (μM) |
| 1 | R, S | 2-Me | 2- | 20.1 ± 1.1 | >50 |
| | | | naphthyl | | |
| 1a | S | 2-Me | 2- | >200 | NA |
| | | | naphthyl | | |
| 2 | R | 2-Me, | 1- | 0.46 ± 0.03 | 6.0 ± 0.1 |
| | | 5-CH₂NH₂ | naphthyl | | |
| 1b | R | 2-Me | 2- | 8.7 ± 0.7 | >50 |
| | | | naphthyl | | |
| 3 | R | 2-Cl | 2- | 14.5 ± 0.9 | >50 |
| | | | naphthyl | | |
| 4 | R | 2-Et | 2- | >200 | NA |
| | | | naphthyl | | |
| 5a | R | 3-Me | 2- | 14.8 ± 5.0 | >50 |
| | | | naphthyl | | |
| 5h | R | 2-Me | 1- | 2.3 ± 0.1 | 10.0 ± 1.2 |
| | | | naphthyl | | |
| 5i | R | 2-Me, 5-NO₂ | 2- | 7.3 ± 0.9 | >50 |
| | | | naphthyl | | |
| 24 | R | 2-Me, 5-NH₂ | 1- | 0.56 ± 0.03 | 14.5 ± 0.8 |
| | | | naphthyl | | |
| 25 | R | 2-Me, 5-NHAc | 1- | 2.64 ± 0.04 | 13.1± 0.7 |
| | | | naphthyl | | |
| 29 | di-Me | 2-Me, 5-NH₂ | 1- | 11.1 ± 1.3 | >50 |
| | | | naphthyl | | |
| 47 | R | 2-Me, | 1- | 4.8 ± 0.4 | >50 |
| | | 5-CH₂NHBoc | naphthyl | | |
| 49 | R | 2-Me, | 1- | 1.3 ± 0.1 | 5.2± 0.3 |
| | | 5-CH₂NHMe | naphthyl | | |
| 50 | R, S | 2-Me, 5-NH₂ | 4- | 1.85 | NA |
| | | | quinolinyl | | |
| 51 | R, S | 2-Me, 4-NO₂ | 4- | 3.5 | NA |
| | | | quinolinyl | | |
| 52 | R, S | 2-Me, 4-NH₂ | 4- | 2.3 | NA |
| | | | quinolinyl | | |
| 53 | R, S | 2-Me, 5-CN | 4- | NA | >50 |
| | | | quinolinyl | | |
| 54 | R, S | 2-Me, | 4- | NA | 16.6 |
| | | 5-CH₂OH | quinolinyl | | |

IC₅₀ = enzyme inhibitory activity.

The data in the Table indicate that PLpro inhibitors have antiviral activity against SARS coronavirus. The asterisk indicates the position of the chiral center. IC⁵⁰ values represent inhibitory activity of PLpro in vitro; >200 indicates IC⁵⁰ value not calculable based on highest concentration tested (200 uM);. EC⁵⁰ values represent antiviral activity of the compounds against SARS-CoV. >50: EC⁵⁰ value not calculable based on highest concentration tested (50 uM); NA: not assayed.

Compound 1 contains a stereogenic center adjacent to the carboxamide moiety, consequently, both the (S) enantiomer, 1a, and (R) enantiomer, 1b, were synthesized to determine the stereoselectivity of PLpro. At a concentration of 100 μM, the (S) enantiomer was found to have only slight inhibitory activity (14%) whereas the (R) enantiomer inhibited PLpro activity over 90%, with an IC₅₀ value of 8.7±0.7 μM (Table 5). Without being bound by theory, it is believed herein that the stereochemical preference for the (R) over the (S) enantiomer is consistent for a protein that fits the four-location model for stereospecific recognition (Mesecar A D & Koshland D E, Jr. (2000) A New Model For Protein Stereospecificity. Nature 403(6770):614-615).

The substitution of a chlorine atom (compound 3), resulted in a 2-fold decrease in inhibitory potency (IC₅₀=14.5±0.9 μM) compared to 1b, and the substitution of a larger ethyl group (compound 4), showed much lower activity (IC₅₀>100 μM). Without being bound by theory, it is believed herein that the optimum size of a substituent at the corresponding position is a methyl group. The effect of changing the orientation of the relatively bulky naphthalene group (e.g. Ar¹) is also described herein. Replacing the 2-naphthyl group of 1b with a 1-naphthyl to form compound 5h resulted in a 4-fold increase in inhibitory potency (IC₅₀=2.3 μM). The addition of a second and additional substitutions R^{a} of the phenyl group, such as the ortho-methyl benzene ring of compound 5h is also described herein. Addition of an NHAc group (compound 25), did not substantially affect the IC₅₀ value (2.6 μM±0.1 μM) compared to 5h, whereas the addition of a nitro group, 5i, decreased activity nearly 3-fold. In contrast, the addition of an amino group at the same position (compound 24) increased the inhibitory potency almost 4-fold (IC₅₀=0.6±0.1 μM). Without being bound by theory, it is believed that an additional hydrogen bond may be formed in the enzyme-inhibitor complex when an active hydrogen functional group is included in R^{a}.

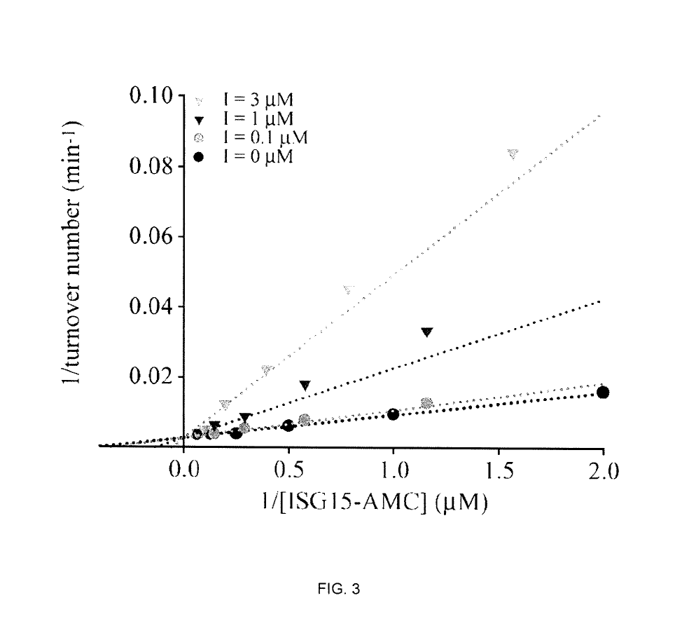

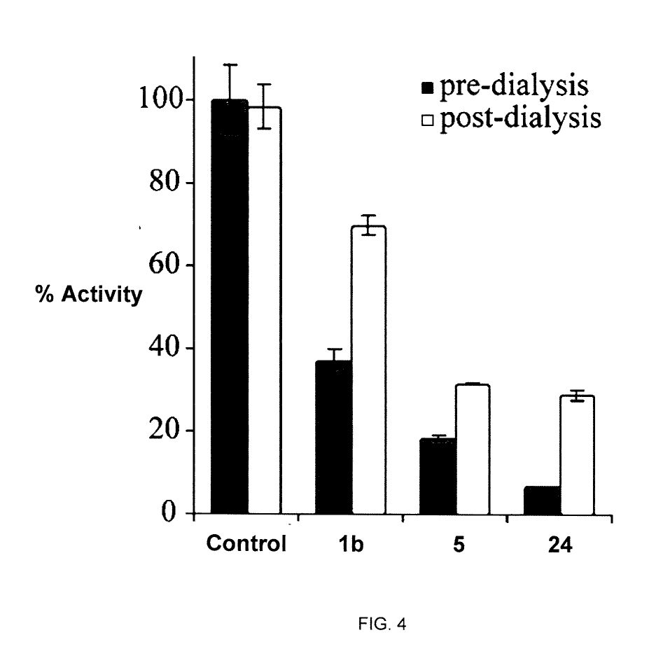

Mechanism of Inhibition. To characterize the mechanism of inhibition of the compounds described herein, kinetic and biochemical studies of the enzyme-inhibitor complexes with compound 24 were performed. A kinetic study of PLpro activity, in which the concentration of its optimal substrate, ISG15-AMC, was varied relative to fixed concentrations of inhibitor, reveals that 24 is a potent, competitive inhibitor of PLpro with a K value of 0.49±0.08 μM (FIG. 3). Progress curve analysis also suggested that the inhibitor is non-covalent. To further probe for noncovalent inhibition of PLpro, PLpro was incubated for 1 hour with 12 μM compound 24 (>20-fold the Kᵢ value), and the resulting complex was dialyzed to allow the inhibitor to diffuse away and therefore restore enzymatic activity (FIG. 4). The results for compounds 2 and 5 are also shown in FIG. 4. Approximately 25% of the PLpro activity was recovered after 3 hours of dialysis compared to a recovery of 100% for the enzyme without inhibitor. Without being bound by theory, it is suggested herein that the inability to fully recover PLpro activity after 3 hours could be either a result of a slow off-rate of the inhibitor from the PLpro-24 complex or a result of covalent modification of the active site cysteine by a direct reaction with the inhibitor or by indirect oxidation. Without being bound by theory, it is believed that, because 24 has no apparent thiol-reactive groups, the inability to recover enzymatic activity is likely a result of both mechanisms, a slow off-rate of inhibitor and oxidation of the cysteine, despite the use of reducing agents throughout all studies. Evidence for cysteine oxidation, revealed in the structural studies, is described hereinbelow.

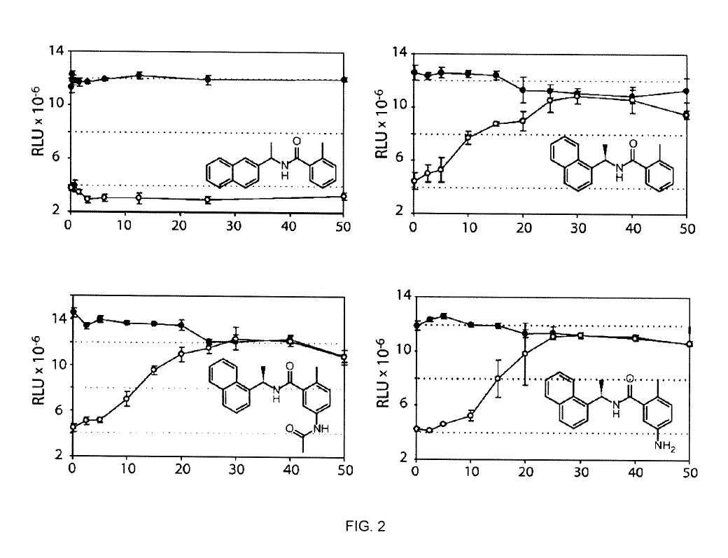

SARS-CoV Antiviral Activity. The antiviral activity of the PLpro inhibitor compounds is described herein. Several compounds were assayed for their ability to rescue cell culture from SARS-CoV infection. The viability of virus-infected Vero E6 cells as a function of inhibitor concentration was measured relative to mock-infected cells using a luminescence assay which allows for the evaluation of both inhibitor efficacy and cytotoxicity (FIG. 2). Compounds 24, 5h, and 25 display significant antiviral activity with EC₅₀ values ranging from 10 to 15 μM without toxicity up to the highest concentration tested (Table 5, FIG. 2). Without being bound by theory, it is believed herein that the increasing antiviral potency correlates with the in vitro inhibition of PLpro, suggesting that the compounds work directly on the enzyme in cells.

Structural Basis for Potent Inhibition of SARS-CoV PLpro Revealed by X-ray Crystallography. To better understand the molecular basis for inhibition of PLpro by the compounds described herein, the X-ray structure of the PLpro-compound 24 complex to a resolution of 2.5 Å was determined (see TABLE 11). The structure reveals unambiguous electron density for the inhibitor, which binds in a cleft leading to the active site.

The inhibitor is well-removed from the catalytic triad and is instead bound within the S3 and S4 subsites of PLpro. Without being bound by theory, it is believed that the interaction between 24 and PLpro is stabilized through a pair of hydrogen bonds and a series of hydrophobic interactions stemming from residues lining the pocket. Specifically, the amide group of the inhibitor forms hydrogen bonds with the side-chain of D165 and the backbone nitrogen of Q270. D165 is highly conserved among the ubiquitin specific protease (USP) family of deubiquitinating enzymes (Quesada V, et al. (2004) Cloning And Enzymatic Analysis Of 22 Novel Human Ubiquitin-Specific Proteases. Biochem Biophys Res Commun 314(1):54-62) and among most coronaviral papain-like proteases (Barretto N, et al. (2005); Sulea T, Lindner H A, Purisima E O, & Menard R (2006) Binding Site-Based Classification Of Coronaviral Papain-Like Proteases. Proteins 62(3):760-775). Several structural studies of USP's have revealed that this aspartic acid residue hydrogen bonds with the backbone of ubiquitin molecules at the P4 position, an interaction without being bound by theory, is believed to be important for ligand stabilization (Hu M, et al. (2005) Structure And Mechanisms Of The Proteasome-Associated Deubiquitinating Enzyme USP14. Embo J 24(21):3747-3756; Hu M, et al. (2002) Crystal Structure Of A UBP-Family Deubiquitinating Enzyme In Isolation And In Complex With Ubiquitin Aldehyde. Cell 111(7):1041-1054; Renatus M, et al. (2006) Structural Basis Of Ubiquitin Recognition By The Deubiquitinating Protease USP2. Structure 14(8):1293-1302).

Aside from the two aforementioned hydrogen bonds, the majority of contacts between PLpro and inhibitor 24 are hydrophobic in nature. The 1-naphthyl group is partly solvent-exposed but forms hydrophobic interactions with the aromatic rings of Y265 and Y269 and with the side-chains of P248 and P249. These residues line the pocket and accommodate the leucine at the P4 position of PLpro substrates (Ratia K, et al. (2006)) (FIG. 5B). Without being bound by theory, it is believed that the (R)-methyl group, attached to a stereogenic atom of the inhibitor, points directly into the interior of the protein between Y265 and T302, where it is accommodated by a cavity that is mostly polar in nature. The positions of three bound water molecules in this cleft, two of them are buried deep in the pocket (P5), whereas the third one lies in a groove between residues Lys158 and Glu168, suggest the potential for extending the (R)-methyl group further into the pocket by the addition of polar substituents.

The disubstituted benzene ring at the opposite end of the inhibitor occupies the putative P3 position of bound substrate. The benzene ring stacks against the aliphatic portions of G164, D165 and Q270, whereas the ortho-methyl substituent points into the floor of the cavity, which is lined by the side-chains of Y265, Y274 and L163 (FIG. 5C). The other ring substituent, 5-NH₂, of compound 24, extends from the opening of the cleft where it is surrounded by a series of polar groups, including the side-chain oxygens of Q270 and E168 and the hydroxyl of Y269, any of which could serve as a hydrogen bond acceptor.

Comparison of the unbound and inhibitor-bound structures reveals two significant conformational differences, both, without being bound by theory, it is believed are induced by inhibitor binding. In the apoenzyme structure, a highly mobile loop hinged by two glycine residues (G267-G272) is positioned in different conformations in each of the three monomers of the asymmetric unit (Ratia K, et al. (2006)). Movements of homologous loops in the deubiquitinating enzymes USP14 and HAUSP upon substrate binding have been observed (Hu M, et al. (2005) and Hu M, et al. (2002)). Without being bound by theory, it is believed that, with PLpro, inhibitor binding induces closure of this loop such that it clamps the inhibitor to the body of the protein. The side chains of Y269 and Q270 become well-defined and reorient to close over the inhibitor, while the main chain of the loop moves to within hydrogen bonding distance of the carbonyl at the center of the inhibitor. Additional movements are observed upon inhibitor binding whereby the side chain of L163 moves to cradle the ortho-methyl of the benzene ring while simultaneously blocking access to the catalytic triad. The plasticity of this region, especially the G267-G272 loop, which is a highly variable region both in length and sequence among papain-like proteases, may account for the range of substrates recognized by these enzymes.

An energy-minimized computer model of compound 2 in the compound 24-inhibited SARS-CoV PLpro active site was constructed. Without being bound by theory, it is believed that the model reveals that the 5-methylamine substituent of inhibitor 2 may be involved in hydrogen bonding with the side chain of residues Gln270 and Tyr269. Similar to the crystal structure conformation of compound 24, compound 2 appears to be anchored in the site by two effective hydrogen bonds made between the carboxamide group and residues Asp165 and Gln 270. Without being bound by theory, it is believed that the three conserved water molecules found in both the crystal structure of the PLpro-24 complex and the crystal structure of the apoenzyme influence the position of the naphthyl ring of the inhibitor, causing it to be flipped upward from the P5 site into a position where it can interact with the flexible peptide loop.

In contrast to the motions observed outside of the catalytic center, the residues of the catalytic triad of PLpro (C112, H273, D287) undergo limited movement between the bound and unbound conformations. A significant amount of residual electron density surrounding the sulfur atom of the catalytic cysteine was observed. Modeling and refinement of this density against a fully-oxidized sulfur atom was consistent with a sulfonic acid moiety, versus sulfinic or sulfenic acids. This observation likely explains the inability of PLpro to regain full activity after incubation with inhibitor over extended periods of time. The presence of reducing agents in solution most likely helps to maintain the active site cysteine of PLpro in a reduced state. Without being bound by theory, it is believed possible that, upon inhibitor binding, loop closure may restrict access to the cysteine by reducing agents but still allow for oxidation, thereby generating an inactive enzyme. A similar mechanism has been proposed for protein tyrosine phosphatase 1B inhibitors (van Montfort R L, Congreve M, Tisi D, Carr R, & Jhoti H (2003) Oxidation State Of The Active-Site Cysteine In Protein Tyrosine Phosphatase 1B. Nature 423(6941):773-777).

Inhibitor Specificity. Structural and functional studies have revealed that PLpro is homologous to human deubiquitinating enzymes and is capable of cleaving ubiquitin and ubiquitin-like modifiers such as ISG15 (Lindner H A, et al. (2005); Ratia K, et al. (2006); Barretto N, et al. (2005); Sulea T, Lindner H A, Purisima E O, & Menard R (2005); Lindner H A, et al. (2007) Selectivity In ISG15 And Ubiquitin Recognition By The SARS Coronavirus Papain-Like Protease. Arch Biochem Biophys 466(1):8-14). Since there are over 50 putative de-ubiquitinating enzymes in humans that are also cysteine proteases (Daviet L & Colland F (2008) Targeting Ubiquitin Specific Proteases For Drug Discovery. Biochimie 90(2):270-283), it is believed herein that any inhibitors being developed are advantageously selective for PLpro. To test the selectivity of the lead inhibitor against PLpro, the inhibitory activities of 24 against a series of cysteine proteases, including the human deubiquitinating enzymes HAUSP, USP18, UCH-L1, UCH-L3, and NL63-CoV papain-like protease 2 (PLP2) from the human coronavirus NL63, were tested. The results are shown in Table 6.

TABLE 6
IC₅₀ values (μM)

| | | NL63 | | | | |
|---|---|---|---|---|---|---|
| Compound | PLpro | PLP2 | hUCH-L1 | hUCH-L3 | HAUSP | hUSP18 |
| 5h | 2.3 | >100 | >100 | >100 | NA | NA |
| 25 | 2.6 | >100 | >100 | >100 | NA | NA |
| 24 | 0.6 | >100 | >100 | >100 | >100 | >100 |

NA indicates that inhibition of the enzyme was not assayed

The results indicate that compound 24 is selective for SARS-CoV PLpro. IC₅₀ values of compounds 5h, 25, and 24 are listed for PLpro and 5 other papain-like proteases.

Although the tested enzymes share similar active site architectures to PLpro, it was observed herein that none of these DUB-like enzymes were inhibited by 24. Structural alignment of the PLpro-24 complex with one of SARS-CoV PLpro's closest structural neighbors, HAUSP, reveals that at least two residues of HAUSP, F409 and K420, sterically clash with the inhibitor binding site.

Based on a structural alignment of 54 human ubiquitin-specific proteases (USPs), these two residues are >80% identical among family members (Quesada V, et al. (2004); Renatus M, et al. (2006)), suggesting that compound 24 is unlikely to inhibit other human USPs.

### Methods

PLpro Purification and Kinetic Assays. Untagged, native SARS-CoV PLpro (polyprotein residues 1541-1855) was expressed and purified to >99% purity as previously described (Barretto N, et al. (2005)). Kinetic assay development was first optimized in a 96-well plate format to establish suitable assay conditions and incubation times. The fluorogenic peptide substrate, Arg-Leu-Arg-Gly-Gly-AMC (SEQ ID NO: 4) (RLRGG-AMC), was purchased from Bachem Bioscience, Inc. PLpro activity as a function of substrate concentration was measured to determine a suitable sub-saturating, substrate concentration for HTS. Enzyme concentration and incubation time with substrate were optimized to yield a linear response in a 6-minute time frame. Bovine serum albumin (BSA) was included in the assay to stabilize PLpro, to prevent the adsorption of PLpro to the assay plate, and to reduce the effects of promiscuous inhibitors. Reducing agent, 5 mM dithiothreitol (DTT) in this case, was included in all assays to eliminate cysteine-reactive compounds.

Primary HTS Screening. A compound library consisting of 50,080 structurally diverse small molecules was purchased from ChemBridge Corporation (San Diego, Calif.) and maintained as 10 mM stock solutions dissolved in dimethylsulfoxide (DMSO) and stored desiccated at −20° C. The automated primary screen was performed on a Tecan Freedom EVO 200 robot equipped with a Tecan 3×3 mounted 96-well dispenser and a 384-pin stainless steel pin tool (V&P Scientific) with a 100 nL capillary capacity. Fluorescence values were measured on an integrated Tecan Genios Pro microplate reader. All assays were performed in duplicate at room temperature, in black flat-bottom 384-well plates (Matrix Technologies) containing a final reaction volume of 50 μL. The assays were assembled as follows: 40 μL of 142 nM PLpro in Buffer A (50 mM HEPES pH 7.5, 0.1 mg/mL BSA, and 5 mM DTT) was dispensed into wells and then incubated with 100 nL of 10 mM inhibitor (20 μM final concentration) for approximately 5 minutes. Reactions were then initiated with 10 μL of 250 μM RLRGG-AMC (SEQ ID NO: 4) in Buffer A, shaken vigorously for 30 s and then incubated for 6 minutes. Reactions were subsequently quenched with 10 μL of 0.5 M acetic acid, shaken for 30 s, and measured for fluorescence emission intensity (excitation λ: 360 nm; emission λ: 460 nm). Each 384-well plate contained 32 positive control wells (100 nL of DMSO replacing 100 nL of inhibitor in DMSO) and 32 negative control wells (assay components lacking PLpro). Due to the low hit rate of compounds displaying significant PLpro inhibition, compounds that showed greater than 35% inhibition were selected for further analysis.

IC₅₀ Value Determination. IC₅₀ measurements were performed by hand, in duplicate, in a 96-well plate format. Buffer, enzyme, and substrate conditions matched those of the primary screen. Reactions containing 50 μM substrate, 2% DMSO, and varying concentrations of inhibitor (0-200 μM)were initiated with the addition of enzyme. Reaction progress was monitored continuously on a Tecan Genios Pro microplate reader (excitation λ: 360 nm; emission λ: 460 nm). Data were fit to the equation : vi=vo/(1+[I]/IC₅₀) using the Enzyme Kinetics module of SigmaPlot (v. 9.01 Systat Software, Inc.) where vi is the reaction rate in the presence of inhibitor, vo is the reaction rate in the absence of inhibitor, and [I] is the inhibitor concentration. Results are shown in the following TABLES 7-10 and in TABLE 5

TABLE 7

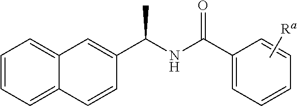

| | | IC₅₀ |
|---|---|---|
| Compound | R^{a} | (μM) |
| 1b | 2-Me | 8.7 ± 0.7 |
| 5a | 3-Me | 14.8 ± 5.0 |
| 5b | 4-Me | 29.1 ± 3.8 |
| 5c | 2-OMe | 90 ± 26 |
| 5d | 3-OMe | 13.5 ± 6.8 |
| 5e | 4-OMe | 149 ± 43 |

TABLE 8

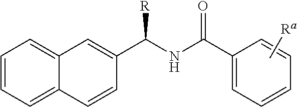

| | | | IC₅₀ |
|---|---|---|---|
| Compound | R | R^{a} | (μM) |
| 1b | Me | 2-Me | 8.7 ± 0.7 |
| 3 | Me | 2-Cl | 14.5 ± 0.9 |
| 4 | Me | 2-Et | >200 |
| 5f | Me | 2,6-diMe | 12.1 ± 0.7 |
| 5g | Me | 2-OH | >200 |
| 9 | Me | 4-NH₂ | 46.1 ± 13.0 |
| 8 | Me | 4-Boc-NH | >200 |
| 14 | Et | (racemic) 2-Me | >200 |
| 17 | Ph | (racemic) 2-Me | >200 |

IC₅₀ = enzyme inhibitory activity.

TABLE 9

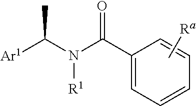

| | | | | IC₅₀ |
|---|---|---|---|---|
| Compound | Ar¹ | R¹ | R^{a} | (μM) |
| 1b | 2-naphthyl | H | 2-Me | 8.7 ± 0.7 |
| 5h | 1-naphthyl | H | 2-Me | 2.3 ± 0.1 |
| 21 | 1-naphthyl | Me | 2-Me | 22.6 ± 6.9 |
| 23 | 1-naphthyl | H | 4-NH₂ | 24.8 ± 1.0 |
| 24 | 1-naphthyl | H | 2-Me and 5-NH₂ | 0.56 ± 0.03 |
| 25 | 1-naphthyl | H | 2-Me and 5-NHAc | 2.64 ± 0.04 |

TABLE 10

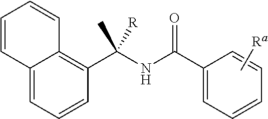

| | | | IC₅₀ |
|---|---|---|---|
| Compound | R | R^{a} | (μM)a |
| 24 | H | 2-Me and 5-NH₂ | 0.56 ± 0.03 |
| 29 | Me | 2-Me and 5-NH₂ | 11.1 ± 1.3 |
| 33 | H | 2-Me and 5-CN | 5.2 ± 0.5 |
| 40 | H | 2-CH₂OMe and 5-NH₂ | 2.7 ± 0.1 |
| 32 | H | 2-Me and 5-I | 1.4 ± 0.3 |
| 47 | H | 2-Me and 5-CH₂NHBoc | 4.8 ± 0.4 |
| 49 | H | 2-Me and 5-CH₂NHMe | 1.3 ± 0.1 |
| 2 | H | 2-Me and 5-CH₂NH₂ | 0.46 ± 0.03 |

TABLE 9

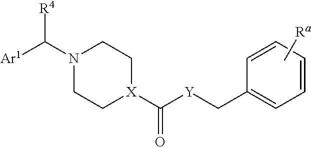

| | | | | | | IC₅₀ | EC₅₀ |
|---|---|---|---|---|---|---|---|
| Cpd | Ar¹ | R⁴ | X | Y | R^{a} | (μM) | (μM) |
| 61 | 2-naphthyl | S—Me | CH | NH | 3-MeO | 13.2 ± 0.6 | 12 |
| 62 | 2-naphthyl | R—Me | CH | NH | 3-OMe | 5.8 ± 0.1 | NA |
| 63 | 1-naphthyl | R—Me | CH | NH | 4-OMe | 0.34 | 10 |
| 64 | 1-naphthyl | R—Me | CH | NH | 3-OMe | 0.34 | 8 |
| 65 | 1-naphthyl | R, S—Me | N | O | 4-OMe | NA | NA |
| 66 | 1-naphthyl | R, S—Me | N | NH | 4-OMe | NA | NA |
| 67 | 1-naphthyl | R—Me | CH | NH | 2-OMe | 1.21 ± 0.04 | NA |
| 68 | 1-naphthyl | S—Me | CH | NH | 3,4-(OCH₂O) | 0.56 | 3 |
| 69 | 1-naphthyl | R—Me | CH | NH | 3,4-(OCH₂O) | 0.32 | 3 |

Reversibility of Inhibition. To test the reversibility of inhibition, 50 nM PLpro was incubated with and without inhibitor (at 20-fold the inhibitor IC₅₀ concentration) in buffer containing 0.05 mg/mL BSA, 50 mM HEPES pH 7.5, 5 mM DTT, and 1% DMSO in a final volume of 3 mL, for 1 h at room temperature. 1.5 mL of each sample was then dialyzed against 1 L of dialysis buffer (50 mM HEPES pH 7.5, 5 mM DTT) for 3 h at room temp using 10,000 MWCO Slide-A-Lyzer dialysis cassettes (Pierce). Samples were transferred to 1 L of fresh dialysis buffer each hour. The other 1.5 mL's of each sample (undialyzed samples) were excluded from dialysis but remained at room temperature for the 3 h time period. All samples were assayed for activity following the 3 h incubation in the same manner as employed for IC₅₀ measurements.

PLpro de-ISGylating Assays. PLpro activity with ISG15-AMC (Boston Biochem) was measured in 96-well half volume plates, at 25° C., in buffer containing 50 mM HEPES pH 7.5, 0.1 mg/mL BSA, 5 mM DTT, 2% DMSO, and fixed inhibitor concentrations of 0, 0.1, 1, and 3 μM. Substrate concentration was varied from 0-16 μM, and release of AMC was measured in the same manner as for the IC₅₀ measurements described above. The Ki and mode of inhibition of inhibitor 24 were determined through Lineweaver-Burk analysis of the above data using the Enzyme Kinetics module of SigmaPlot.

Inhibitor Specificity Assays. The specificity of compounds 2b, 5h, and 24 were tested against two human ubiquitin C-terminal hydrolases, UCH-L1 and UCH-L2, the human deubiquitinating enzyme HAUSP, the human de-ISGylating enzyme USP-18, and a coronaviral papain-like protease from HCoV NL63, PLP2. UCH-L1 and UCH-L2 were purchased from Biomol International, HAUSP and USP-18 from Boston Biochem, and PLP2 was purified as previously described (Chen Z, et al. (2007) Proteolytic Processing And Deubiquitinating Activity Of Papain-Like Proteases Of Human Coronavirus NL63. J Virol 81(11):6007-6018). All kinetic assays were performed at 25° C. in 50 mM HEPES pH 7.5, 0.1 mg/mL BSA, and 5-10 mM DTT in a 96-well plate format. Enzymes were assayed in the absence and presence of 100 μM inhibitor, with 100 nM ubiquitin-AMC (Boston Biochem) as substrate (excitation λ: 360 nm; emission λ: 460 nm), with the exception of USP-18, which was assayed with 1 μM ISG15-AMC (Boston Biochem) as substrate. PLpro was assayed under the same conditions, as a control.

SARS-CoV Antiviral Activity Assays. Vero E6 cells were maintained in Minimal Essential Media (MEM) (Gibco) supplemented with 100 U/mL penicillin, 100 g/mL streptomycin (Gibco) and 10% fetal calf serum (FCS) (Gemini Bio-Products). The SARS-CoV Urbani strain used in this study was provided by the Centers for Disease Control and Prevention (Ksiazek T G, et al. (2003) A Novel Coronavirus Associated With Severe Acute Respiratory Syndrome. N Engl J Med 348(20):1953-1966). All experiments using SARS-CoV were carried out in a Biosafety Level 3 facility using approved biosafety protocols. Vero E6 cells were seeded onto flat-bottom, 96-well plates at a density of 9×10³ cells/well. Cells were either mock infected with serum-free MEM or infected with 100 TCID₅₀/well of SARS-CoV Urbani in 100 μL of serum-free MEM and incubated for 1 hour at 37° C. with 5% CO₂. Following the one hour incubation period, the viral inoculum was removed and, 100 μL of MEM supplemented with 2% FCS and containing the inhibitor compound of interest at the desired concentration (serial 2-fold dilutions from 50 μM to 0.1 μM) was added. Cells were incubated for a period of 48 hours at 37° C. with 5% CO₂. Each condition was set up in triplicate and antiviral assays were performed independently on at least two separate occasions. Cell viability was measured 48 hours post infection using the CellTiter-Glo Luminescent Cell Viability Assay (Promega), according to manufacturer's recommendations. Cell viability for the CellTiter-Glo Luminescent Cell Viability Assay was measured as luminescence and output expressed as relative luciferase units (RLU).

High-throughput screen hit confirmation (secondary screening) 17 compounds from the initial screen were repurchased from ChemBridge Corporation and maintained as 30 mM stocks in DMSO. Compounds were tested in triplicate, in a dose-dependent assay, using 384-well plates. Assays were performed as in the primary screen, using a range of inhibitor concentrations (142.8, 71.4, 35.7, 17.9, 8.9, 4.5, and 2.2 μM) in both the ab-sence and presence of 0.01% Triton-X to eliminate promiscuous inhibitors (Feng B Y & Shoichet B K (2006) A Detergent-Based Assay For The Detection Of Promiscuous Inhibitors. Nat Protoc 1(2):550-553). To eliminate compounds that interfered with AMC fluorescence and thus produced false positives, the fluorescence of free AMC was measured in the presence of 50 μM inhibitor. Inhibitors that produced a significant decrease in AMC fluorescence were eliminated from further screening.

Crystallization, X-ray Data Collection, and Structure Refinement The complex of inhibitor 24 with PLpro was crystallized by vapor diffusion in a sitting-drop format following a 16 h incubation of 8 mg/mL PLpro (in 20 mM Tris pH 7.5, 10 mM DTT) with 2 mM inhibitor at 4° C. Immediately prior to crystallization, the sample was clarified by centrifugation. A 1 μL volume of the enzyme-inhibitor solution was then mixed with an equal volume of well solution containing 1M LiCl, 0.1M MES pH 6.0, and 30% PEG 6,000 and equilibrated against well solution at 20° C. Prior to data collection, crystals were soaked in a cryosolution containing well solution, 400 μM inhibitor, and 16% glycerol. Crystals were flash-frozen in liquid nitrogen and then transferred into a dry nitrogen stream at 100 K for X-ray data collection. The data set of the complex was collected at the Southeast Regional Collaborative Access Team (SERCAT) 22-BM beamline at the Advanced Photon Source, Argonne National Laboratory. Data were processed and scaled by using the HKL2000 program suite (Otwinowski Z & Minor W (1997) Methods in Enzymology, Volume 276: Macromolecular Crystallography. Methods in Enzymology, (Academic Press, New York), Vol 276: Macromolecular Crystallography, pp 307-326). Crystals belonged to the space group I222, with one monomer in the asymmetric unit. The inhibitor-complexed structure was solved by molecular replacement using the SARSCoV PLpro apoenzyme structure (PDB entry: 2FE8) (Ratia K, et al. (2006)) as a search model in the AMoRe program (33) of the CCP4 suite (Collaborative Computational Project N (1994) “The CCP4 Suite: Programs for Protein Crystallography”. Acta Cryst. D50:760-763), and the structure was refined to 2.5 Å using CNS (Brunger A T, et al. (1998) Crystallography & NMR system: A New Software Suite For Macromolecular Structure Determination. Acta Crystallogr D Biol Crystallogr 54 (Pt 5):905-921). Final X-ray data collection and refinement statistics are given in TABLE 5.

TABLE 11
Data Collection and Refinement Statistics

| Data set: | PLpro-24 | |
|---|---|---|
| Data Collection | | |
| Space group | I222 | |
| Unit Cell dimensions: | | |
| a, b, c (Å) | 71.70, 90.68, 109.54 | |
| Resolution (Å) | 50-2.50 | |
| No. Reflections Observed | 88,864 | |
| No. Unique Reflections | 12,480 | |
| R_{merge} (%) | 10.7 | (42.4)* |
| I/σI | 23.7 | (2.8)* |
| % Completeness | 98.6 | (93.6)* |
| Refinement | | |
| Resolution Range | 20-2.50 | |
| No. Reflections in Working Set | 11,874 | |
| No. Reflections in Test Set | 606 | (4.8%) |
| R_{cryst} (%) | 19.6 | |
| R_{free} (%) | 26.1 | |
| Average B-factor (Å²) | 48.8 | |
| RMSD from ideal geometry: | | |
| Bond Lengths (Å) | 0.007 | |
| Bond Angles (degrees) | 1.2 | |
| Ramachandran Plot | | |

| | |
|---|---|
| Most favored (%) | 86.7 |
| Allowed (%) | 13.3 |
| Disallowed (%) | 0 |

*Data for the last resolution shell (2.59-2.50 Å) are shown in parentheses.

### General

¹HNMR and ¹³CNMR spectra were recorded on Varian Oxford 300 and Bruker Avance 400 spectrometers. Optical rotations were recorded on Perkin-Elmer 341 polarimeter. Anhydrous solvent was obtained as follows: dichloromethane by distillation from CaH₂, THF by distillation from Na and benzophenone. All other solvents were reagent grade. Column chromatography was performed with Whatman 240-400 mesh silica gel under low pressure of 3-5 psi. TLC was carried out with E. Merck silica gel 60-F-254 plates. Purity of all test compounds was determined by HRMS and HPLC analysis in two different solvent systems. All test compounds showed 95% purity.

HPLC system used: Agilgent 1100 series. Column and flow rate employed: XDB-C18, 5 μm 4.6×150 mm and 1.5 mL/min. Solvent system A=linear gradient from 25% acetonitrile, 75% water to 90% acetonitrile, 10% water in 15 min. Solvent system B=linear gradient from 30% methanol, 70% water to 100% methanol in 18 min. Solvent system C=linear gradient from 20% acetonitrile, 80% 25 mM NH₄OAc in water (pH 4.8) to 80% acetonitrile, 20% 25 mM NH₄OAc in water (pH 4.8) in 15 min.

| | Solvent | Retention Time | Purity |
|---|---|---|---|
| Inhibitor | system | (min) | (%) |
| 5a | A | 10.9 | 99.3 |
| 5b | A | 10.8 | 99.7 |
| 5c | A | 11.0 | 98.3 |
| 5d | A | 10.2 | 99.6 |
| 5e | A | 9.9 | 98.0 |
| 5f | A | 10.7 | 96.7 |
| 5g | A | 11.6 | 98.7 |
| 5h | A | 10.5 | 99.7 |
| 5i | A | 10.7 | 98.5 |
| 8 | A | 11.5 | 96.5 |
| 9 | A | 7.9 | 99.3 |
| 14 | A | 11.3 | 99.8 |
| 17 | A | 12.5 | 99.7 |
| 21 | A | 12.4 | 99.9 |
| 23 | A | 7.8 | 99.3 |
| 24 | A | 8.1 | 99.2 |
| 25 | A | 7.7 | 98.9 |
| 29 | A | 8.6 | 98.2 |
| 32 | B | 13.1 | 96.0 |
| 33 | B | 11.2 | 99.9 |
| 40 | B | 10.8 | 95.3 |
| 47 | A | 12.9 | 99.8 |
| 49 | C | 5.0 | 98.6 |
| 2 | C | 4.7 | 98.3 |

### EXAMPLES

The general procedure for amide coupling is demonstrated by the following example.

2-Methyl-N—[(R)-1-(1-naphthyl)ethyl]benzamide (5h). To a solution of o-toluic acid (16.2 mg, 0.12 mmol), N-(3-dimethylaminopropyl)-N⁰-ethylcarbodiimide hydrochloride (EDCI) (29.1 mg, 0.15 mmol), and 1-hydroxybenzotriazole hydrate (HOBT) (20.5 mg, 0.15 mmol) in dry CH₂Cl₂was added a solution of (R)-(+)-1-(1-naphthyl)ethylamine 18 (20 mg, 0.12 mmol) and diisopropylethylamine (81.4 μL, 0.47 mmol) in dry CH₂Cl₂ at 0° C. under argon atmosphere and it was allowed to stir for 15 h at 23° C. The reaction mixture was quenched with water and extracted with CH₂Cl₂. The organic layers were dried over Na₂SO₄ and concentrated under reduced pressure. The residue was purified by silica gel column chromatography to furnish compound 51 (33 mg, 98%) as a white solid, R_{f}=0.34 (hexane:EtOAc=3:1), [α]₂₀^{D}−50.0 (c=1, CHCl₃). ¹HNMR (400 MHz, CDCl₃): δ 8.24 (d, 1H, J=8.5 Hz), 7.89 (d, 1H, J=8.0 Hz), 7.82 (d, 1H, J=8.0 Hz), 7.60-7.51 (m, 3H), 7.46 (dd, 1H, J=7.6 and 7.7 Hz), 7.27-7.24 (m, 2H), 7.17 (d, 1H, J=7.7 Hz), 7.11 (dd, 1H, J=7.6 and 8.0 Hz), 6.15-6.07 (m, 2H), 2.44 (s, 3H), 1.79 (d, 3H, J=6.4 Hz). ¹³C NMR (100 MHz, CDCl₃): δ 168.9, 137.9, 136.3, 136.0, 133.9, 131.1, 130.9, 129.7, 128.7, 128.4, 126.5, 126.5, 129.5, 125.6, 125.1, 123.5, 122.5, 44.8, 20.5, 19.7. MS (EI): m/z 289.20 [M]⁺. HRMS (EI), calcd for C₂₀H₁₉NO 289.1467, found [M]⁺ 289.1468.

### EXAMPLE

2-Methyl-N—[(S)-1-(2-naphthyl)ethyl benzamide (1a). white solid (yield: 95%), R_{f}=0.34 (hexane:EtOAc=3:1), [α]²⁰_{D}−46.2 (c=1, CHCl₃); ¹H NMR (300 MHz, CDCl₃): δ 7.94-7.89 (m, 4H), 7.60-7.53 (m, 3H), 7.45-7.33 (m, 2H), 7.29-7.26 (m, 2H), 6.29 (d, 1H, J=7.5 Hz), 5.61-5.54 (m, 1H), 2.51 (s, 3H), 1.75 (d, 3H, J=6.3 Hz); ¹³C NMR (75 MHz, CDCl₃): δ 169.2, 140.4, 136.4, 136.0, 133.3, 132.7, 130.9, 129.8, 128.5, 127.8, 127.6, 126.6, 126.2, 125.9, 125.7, 124.7, 124.5, 49.0, 21.7, 19.7.

### EXAMPLE

2-Methyl-N—[(R)-1-(2-naphthyl)ethyl]benzamide (1b). white solid (yield: >99%), R_{f}=0.34 (hexane:EtOAc=3:1), [α]²⁰_{D}+45.9 (c=1, CHCl₃); ¹H NMR (300 MHz, CDCl₃): δ 7.94-7.90 (m, 4H), 7.61-7.53 (m, 3H), 7.46-7.34 (m, 2H), 7.30-7.26 (m, 2H), 6.27 (d, 1H, J=8.1 Hz), 5.62-5.52 (m, 1H), 2.52 (s, 3H), 1.76 (d, 3H, J=6.9 Hz); ¹³C NMR (75 MHz, CDCl₃): δ 169.2, 140.4, 136.4, 136.0, 133.3, 132.7, 130.9, 129.8, 128.5, 127.8, 127.6, 126.6, 126.2, 125.9, 125.7, 124.7, 124.5, 49.0, 21.7, 19.7.

### EXAMPLE

2-Chloro-N—[(R)-1-(2-naphthyl)ethyl]benzamide (3). white solid (yield: 96%), R_{f}=0.26 (hexane:EtOAc=3:1), [α]²⁰_{D}+27.4 (c=1, CHCl₃); ¹H NMR (300 MHz, CDCl₃): δ 7.92-7.88 (m, 4H), 7.68 (dd, 1H, J=1.8 and 6.9 Hz), 7.60-7.51 (m, 3H), 7.46-7.31 (m, 3H), 6.76 (d, 1H, J=7.8 Hz), 5.60-5.51 (m, 1H), 1.75 (d, 3H, J=6.6 Hz); ¹³C NMR (75 MHz, CDCl₃): δ 165.6, 140.0, 135.0, 133.2, 132.7, 131.1, 131.0, 130.5, 130.1, 128.4, 127.8, 127.5, 127.0, 126.1, 125.8, 124.7, 124.6, 49.5, 21.5.

### EXAMPLE

2-Ethyl-N—[(R)-1-(2-naphthyl)ethyl]benzamide (4). white solid (yield: >99%), R_{f}=0.37 (hexane:EtOAc=3:1), [α]²⁰_{D}++50.0 (c=1, CHCl₃); ¹H NMR (300 MHz, CDCl₃): δ 7.94-7.91 (m, 4H), 7.61-7.53 (m, 3H), 7.45-7.41 (m, 2H), 7.34-7.26 (m, 2H), 6.24 (d, 1H, J=8.1 Hz), 5.63-5.53 (m, 1H), 2.88 (q, 2H, J=7.5 Hz), 1.76 (d, 3H, J=6.3 Hz), 1.30 (t, 3H, J=7.5 Hz); ¹³C NMR (75 MHz, CDCl₃): δ 169.3, 142.3, 140.3, 136.1, 133.3, 132.7, 129.9, 129.4, 128.5, 127.8, 127.6, 126.6, 126.2, 125.9, 125.7, 124.6, 124.5, 49.0, 26.3, 21.6, 15.8.

### EXAMPLE

3-Methyl-N—[(R)-1-(2-naphthyl)ethyl]benzamide (5a). The title compound was obtained as described in the general procedure in 92% yield (white solid). R_{f}=0.35 (hexane:EtOAc=3:1), [α]₂₀^{D}+39.5 (c=1, CHCl₃). ¹H NMR (300 MHz, CDCl₃): δ 7.83-7.79 (m, 4H), 7.60-7.44 (m, 5H), 7.27 (d, 2H, J=5.4 Hz), 6.51 (d, 1H, J=6.9 Hz), 5.53-5.44 (m, 1H), 2.35 (s, 3H), 1.67 (d, 3H, J=6.6 Hz). ¹³C NMR (75 MHz, CDCl₃): δ 166.8, 140.5, 138.3, 134.5, 133.3, 132.7, 132.2, 128.5, 128.4, 127.9, 127.6, 127.6, 126.2, 125.8, 124.8, 124.6, 123.9, 49.1, 21.5, 21.3. MS (EI): m/z 289.15 [M]⁺. HRMS (EI), calcd for C₂₀H₁₉NO 289.1467, found [M]⁺ 289.1468.

### EXAMPLE

4-Methyl-N—[(R)-1-(2-naphthyl)ethyl]benzamide (5b). The title compound was obtained as described in the general procedure in >99% yield (white solid). R_{f}=0.32 (hexane:EtOAc=3:1), [α]₂₀^{D}+19.7 (c=1, CHCl₃). ¹H NMR (300 MHz, CDCl₃): δ 7.82-7.79 (m, 4H), 7.68 (d, 2H, J=8.1 Hz), 7.50-7.42 (m, 3H), 7.17 (d, 2H, J=7.5 Hz), 6.59 (d, 1H, J=6.9 Hz), 5.52-5.42 (m, 1H), 2.36 (s, 3H), 1.64 (d, 3H, J=6.9 Hz). ¹³C NMR (75 MHz, CDCl₃): δ 166.5, 141.8, 140.6, 133.3, 132.7, 131.6, 129.1, 128.5, 127.9, 127.6, 126.9, 126.2, 125.8, 124.8, 124.6, 49.1, 21.6, 21.4. MS (EI): m/z 289.10 [M]⁺. HRMS (EI), calcd for C₂₀H₁₉NO 289.1467, found [M]⁺ 289.1469.

### EXAMPLE

2-Methoxy-N—[(R)-1-(2-naphthyl)ethyl]benzamide (5c). The title compound was obtained as described in the general procedure in >99% yield (white solid). R_{f}=0.23 (hexane:EtOAc=3:1), [α]₂₀^{D}−30.7 (c=1, CHCl₃). ¹H NMR (300 MHz, CDCl₃): δ 8.28 (d, 1H, J=7.8 Hz), 8.22 (dd, 1H, J=1.8 and 8.1 Hz), 7.52 (dd, 1H, J=1.8 and 8.7 Hz), 7.49-7.40 (m, 3H), 7.07 (t, 1H, J=7.7 Hz), 6.95 (d, 1H, J=9.0 Hz), 5.57-5.47 (m, 1H), 3.92 (s, 3H), 1.67 (d, 3H, J=6.3 Hz). ¹³C NMR (75 MHz, CDCl₃): δ 164.4, 157.5, 141.2, 133.4, 132.7, 132.6, 132.3, 128.4, 127.8, 127.6, 126.1, 125.7, 124.7, 124.4, 121.6, 121.3, 111.3, 55.9, 49.1, 22.3. MS (EI): m/z 305.15 [M]+. HRMS (EI), calcd for C₂₀H₁₉NO₂305.1416, found [M]⁺ 305.1414.

### EXAMPLE

3-Methoxy-N—[(R)-1-(2-naphthyl)ethyl]benzamide (5d). The title compound was obtained as described in the general procedure in >99% yield (white solid). R_{f}=0.24 (hexane:EtOAc=3:1), [α]₂₀^{D}+50.0 (c=1, CHCl₃). ¹H NMR (300 MHz, CDCl₃): δ 7.82-7.79 (m, 4H), 7.50-7.44 (m, 3H), 7.38-7.38 (m, 1H), 7.31-7.27 (m, 2H), 7.03-6.97 (m, 1H), 6.57 (d, 1H, J=7.8 Hz), 5.51-5.42 (m, 1H), 3.79 (s, 3H), 1.65 (d, 3H, J=7.2 Hz). ¹³C NMR (75 MHz, CDCl₃): δ 166.4, 159.8, 140.5, 136.0, 133.3, 132.7, 129.5, 128.5, 127.9, 127.6, 126.2, 125.9, 124.7, 124.6, 118.6, 117.7, 112.4, 55.4, 49.3, 21.5. MS (EI): m/z 305.20 [M]⁺. HRMS (EI), calcd for C₂₀H₁₉NO₂ 305.1416, found [M]⁺ 305.1417.

### EXAMPLE

4-Methoxy-N—[(R)-1-(2-naphthyl)ethyl]benzamide (5e). The title compound was obtained as described in the general procedure in >99% yield (white solid). R_{f}=0.20 (hexane:EtOAc=3:1), [α]₂₀^{D}+3.0 (c=1, CHCl₃). ¹HNMR (300 MHz, CDCl₃): δ 7.81-7.73 (m, 6H), 7.49-7.41 (m, 3H), 6.85 (d, 2H, J=8.7 Hz), 6.58 (d, 1H, J=7.8 Hz), 5.50-5.40 (m, 1H), 3.79 (s, 3H), 1.63 (d, 3H, J=6.9 Hz). ¹³C NMR (75 MHz, CDCl₃): δ 166.1, 140.7, 133.3, 132.7, 128.7, 128.4, 127.8, 127.5, 126.7, 126.1, 125.8, 124.8, 124.5, 113.6, 55.3, 49.1, 21.6. MS (EI): m/z 305.15 [M]⁺HRMS(EI), calcd for C₂₀H₁₉NO₂305.1416, found [M]⁺ 305.1419.

### EXAMPLE

2,6-Dimethyl-N—[(R)-1-(2-naphthyl)ethyl]benzamide (5f). The title compound was obtained as described in the general procedure in 94% yield (white solid). R_{f}=0.26 (hexane:EtOAc=3:1), [α]₂₀^{D}+32.9 (c=1, CHCl₃). ¹H NMR (300 MHz, CDCl₃): δ 7.82-7.77 (m, 4H), 7.49-7.43 (m, 3H), 7.13 (dd, 1H, J=7.2 and 8.1 Hz), 6.98 (d, 2H, J=7.5 Hz), 6.17 (d, 1H, J=8.1 Hz), 5.56-5.46 (m, 1H), 2.27 (s, 3H), 1.64 (d, 3H, J=6.3 Hz). ¹³C NMR (75 MHz, CDCl₃): δ 169.3, 140.1, 137.5, 134.1, 133.2, 132.7, 128.6, 128.4, 127.8, 127.5, 127.4, 126.2, 125.9, 124.8, 124.6, 48.6, 21.4, 19.0. MS (EI): m/z 303.05 [M]⁺. HRMS (EI), calcd for C₂₁H₂₁NO 303.1623, found [M]⁺ 303.1624.

### EXAMPLE

2-Hydroxy-N—[(R)-1-(2-naphthyl)ethyl]benzamide (5g). The title compound was obtained as described in the general procedure in 97% yield (white solid). R_{f}=0.49 (hexane:EtOAc=3:1), [α]₂₀^{D}+68.3 (c=1, CHCl₃). ¹H NMR (300 MHz, CDCl₃): δ 12.39 (s, 1H), 7.85-7.80 (m, 4H), 7.51-7.33 (m, 5H), 6.97 (d, 1H, J=8.1 Hz), 6.81-6.76 (m, 2H), 5.49-5.39 (m, 1H), 1.67 (d, 3H, J=7.2 Hz). ¹³C NMR (75 MHz, CDCl₃): δ 169.2, 161.5, 139.8, 134.2, 133.2, 132.7, 128.6, 127.8, 127.6, 126.3, 126.0, 125.4, 124.5, 124.5, 118.6, 118.5, 114.1, 49.1, 21.5. MS (EI): m/z 291.10 [M]⁺. HRMS (EI), calcd for C₁₉H₁₇NO₂291.1259, found [M]⁺ 291.1261.

### EXAMPLE

2-Methyl-5-nitro-N—[(R)-1-(1-naphthyl)ethyl]benzamide (5i). The title compound was obtained as described in the general procedure in 95% yield (white solid). R_{f}=0.24 (hexane:EtOAc=3:1), [α]₂₀^{D}−53.0 (c=1, CHCl₃). ¹H NMR (300 MHz, CDCl₃): δ 8.18 (d, 1H, J=8.1 Hz), 8.11-8.06 (m, 2H), 7.87 (d, 1H, J=8.0 Hz), 7.81 (d, 1H, J=8.0 Hz), 7.60-7.43 (m, 4H), 7.32 (d, 1H, J=8.4 Hz), 6.13-6.10 (bm, 2H), 2.49 (s, 3H), 1.80 (d, 3H, J=6.3 Hz). ¹³C NMR (75 MHz, CDCl₃): δ 166.5, 144.3, 137.8, 137.3, 133.8, 131.9, 131.1, 128.9, 128.8, 126.7, 126.1, 125.2, 124.4, 123.2, 122.7, 122.6, 121.6, 45.2, 20.5, 20.0. MS (EI): m/z 334.20 [M]⁺. HRMS (EI), calcd for C₂₀H₁₈N₂O₃ 334.1317, found [M]⁺ 334.1323.

### EXAMPLE

4-N-tert-Butoxycarbonylaminobenzoic Acid (7). To a solution of 4-aminobenzoic acid 6 (520 mg, 3.8 mmol) in dioxane/H₂O (2:1) (13 mL) was added triethylamine (0.79 mL, 5.7 mmol) and Boc₂O(1.31 mL, 5.7 mmol) at 23° C. and it was allowed to stir for 48 h at same temperature. The solvent was removed under reduced pressure, and 3 M HCl (5 mL) was added dropwise to the residue at 0° C. A precipitate was obtained, collected, washed with water, and dried to give corresponding acid 7 (836 mg, 93%) as slightly yellow solid, R_{f}=0.78 (CH₂Cl₂:Me-OH=9:1). ¹H NMR (400 MHz, CDCl₃): δ 9.25 (brs, 1H), 7.91 (d, 2H, J=8.7 Hz), 7.50 (d, 2H, J=8.7 Hz), 1.51 (s, 9H). ¹³C NMR (100 MHz, CDCl₃): δ 169.7, 154.8, 131.8, 125.3, 118.6, 118.5, 81.3, 28.6. MS (Ei): m/z 237.10 [M]⁺. HRMS (EI), calcd for C₁₂H₁₅NO₄ 237.1001, found [M]⁺ 237.1004.

### EXAMPLE

4-N⁰-tert-Butoxycarbonylamino-N—[(R)-1-(2-naphthyl)ethyl]-benzamide (8). The title compound was obtained as described in the general procedure in 60% yield (white solid). R_{f}=0.76 (CH₂Cl₂:MeOH=9:1), [α]₂₀^{D}−91.6 (c=1, CHCl₃:MeOH=1:1). ¹H NMR (300 MHz, CDCl₃): δ 7.77-7.72 (m, 6H), 7.48-7.37 (m, 5H), 5.40-5.33 (m, 1H), 1.60 (d, 3H, J=6.9 Hz), 1.47 (s, 9H). MS (EI): m/z 390.05 [M]⁺. HRMS (EI), calcd for C₂₄H₂₆N₂O₃ 390.1943, found [M]⁺ 390.1942.

### EXAMPLE

4-Amino-N—[(R)-1-(2-naphthyl)ethyl]benzamide (9). To a solution of Boc 8 (60 mg, 0.15 mmol) in CH₂Cl₂ (4 mL) was added dropwise trifluoroacetic acid (0.6 mL) at 23° C. and it was allowed to stir for 2 h at same temperature. The reaction was concentrated under reduced pressure, and the residue was treated with saturated NaHCO₃ solution. The mixture was extracted with CH₂Cl₂. The organic layers were dried over Na₂SO₄ and concentrated under reduced pressure. The residue was purified by silica gel column chromatography to give compound 9 (44 mg, 99%) as a white solid, R_{f}=0.60 (CH₂Cl₂:MeOH=9:1), [α]^{20D}−58.0 (c=1, CHCl₃:MeOH=4:1). ¹H NMR(300 MHz, CDCl₃): δ 7.82-7.80 (m, 4H), 7.60 (d, 2H, J=8.1 Hz), 7.50-7.41 (m, 3H), 6.63 (d, 2H, J=8.7 Hz), 6.23 (d, 1H, J=6.9 Hz), 5.52-5.42 (m, 1H), 1.66 (d, 3H, J=7.2 Hz). MS(EI): m/z 290.15 [M]⁺. HRMS (EI), calcd for C₁₉H₁₈N₂O 290.1419, found [M]⁺ 290.1424.

### EXAMPLE

1-(2-Naphthyl)propanone (12). To a solution of propionyl chloride 11 (5.1 g, 55 mmol) and aluminum chloride (7.7 g, 58 mmol) in 1,2-dichloroethane (16 mL) was added dropwise a solution of naphthalene 10 (7.9 g, 62 mmol) in 1,2-dichloroethane (16 mL) over 3 h at 35° C. and it was allowed to stir for 1 h. The reaction was added 3MHCl solution at 0° C. and then separated a white solid. The filtrate was washed with water. The organic layer was dried over Na₂SO₄ and concentrated under reduced pressure. The residue was purified by silica gel column chromatography to furnish compound 12 (9.9 g, 98%) as a colorless oil, R_{f}=0.56 (hexane:EtOAc=9:1). ¹H NMR (300 MHz, CDCl₃): δ 8.58 (d, 1H, J=8.7 Hz), 7.94 (d, 1H, J=8.1 Hz), 7.86-7.80 (m, 2H), 7.59-7.42 (m, 3H), 3.04 (q, 2H, J=6.9 Hz), 1.27 (t, 3H, J=6.9 Hz). ¹³C NMR (75 MHz, CDCl₃): δ 205.2, 136.0, 133.8, 132.2, 130.0, 128.3, 127.7, 127.1, 126.3, 125.7, 124.3, 35.2, 8.56. MS (EI): m/z 184.15 [M]⁺. HRMS (EI), calcd for C₁₃H₁₂O 184.0888, found [M]⁺ 184.0890.

### EXAMPLE

2-Methyl-N-[1-(2-naphthyl)propyl]benzamide (14). To a solution of ketone 12 (2.1 g, 11.4 mmol) in MeOH (50 mL) was added ammonium acetate (8.8 g, 0.11 mol) and NaBH₃CN (528 mg, 8.0 mmol) at 23° C. and was stirred for 24 h. Conc. HCl was added until pH<2, and the solvent was removed under reduced pressure. The residue was taken up in water (15 mL) and extracted once with Et₂O. The aqueous layer was brought to pH>12 with solid KOH and extracted with CH₂Cl₂. The organic layers were dried over Na₂SO₄ and concentrated under reduced pressure to give amine 13 as crude compound, MS (EI): m/z 185.20 [M]⁺. HRMS (EI), calcd for C₁₃H₁₅N 185.1204, found [M]⁺ 185.1206. The general coupling procedure was carried out with amine 13 (50 mg, mmol) and o-toluic acid (37.5 mg, mmol) to give inhibitor 14 (21 mg, 2 steps 26%) as a white solid, R_{f}=0.25 (hexane:EtOAc=3:1). ¹HNMR (300 MHz,CDCl₃): δ 7.84-7.78(m, 4H), 7.49-7.44 (m, 3H), 7.35-7.26 (m, 2H), 7.20-7.14 (m, 2H), 6.12 (d,1H, J=8.4 Hz), 5.28-5.20 (m, 1H), 2.39 (s, 3H), 2.04-1.95 (m, 2H), 0.99 (t, 3H, J=7.2 Hz). ¹³C NMR (75 MHz, CDCl₃): δ 169.4, 139.4, 136.6, 136.0, 133.3, 132.7, 130.9, 129.8, 128.5, 127.8, 127.6, 126.5, 126.2, 125.8, 125.7, 125.3, 124.7, 55.2, 29.1, 19.7, 10.9. MS (EI): m/z 303.25 [M]⁺. HRMS (EI), calcd for C₂₁H₂₁NO 303.1623, found [M]⁺ 303.1624.

### EXAMPLE

1-(2-Naphthyl)benzylamine (16). To a solution of naphthylphenylketone 15 (600 mg, 2.6 mmol) in MeOH (15 mL) was added ammonium acetate (2 g, 25.9 mmol) and NaBH₃CN(120 mg, 1.9 mmol) at 23° C. and it was allowed to stir for 24 h. Conc HCl was added until pH<2, and the solvent was removed under reduced pressure. The residue was taken up in water (4 mL) and extracted once with Et₂O. The aqueous layer was brought to pH>12 with solid KOH and extracted with CH₂Cl₂. The organic layers were dried over Na₂SO₄ and concentrated under reduced pressure to give amine 16 (48 mg, 8%) as crude compound, R_{f}=0.53 (CH₂Cl₂:MeOH=4:1). ¹H NMR (300 MHz, CDCl₃): δ 7.74-7.53 (m, 5H), 7.28-7.18 (m, 4H), 7.13-7.01 (m, 3H), 5.14 (s, 1H), 1.89 (bs, 1H). ¹³C NMR (75 MHz, CDCl₃): δ 145.2, 142.8, 133.3, 132.5, 130.0, 128.5, 128.2, 127.9, 127.6, 127.0, 126.0, 125.7, 125.6, 124.9, 59.7. MS (EI): m/z 233.30 [M]₊. HRMS (EI), calcd for C₁₇H₁₅N 233.1204, found [M]⁺ 233.1205.

### EXAMPLE

2-Methyl-N-[1-(2-naphthyl)benzyl]benzamide (17). The title compound was obtained as described in the general procedure in 72% yield (white solid). R_{f}=0.39 (hexane:EtOAc=3:1). ¹H NMR (300 MHz, CDCl₃): δ 7.88-7.80 (m, 5H), 7.55-7.34 (m, 9H), 7.29-7.24 (m, 2H), 6.66 (d, 1H, J=8.4 Hz), 6.57 (d, 1H, J=8.4 Hz). ¹³C NMR (75 MHz, CDCl₃): δ 169.1, 141.3, 138.7, 136.3, 136.0, 133.2, 132.7, 131.1, 130.0, 128.7, 128.7, 128.6, 128.0, 127.6, 127.5, 126.6, 126.3, 126.1, 126.0, 125.7, 125.5, 57.3, 19.8. MS (EI): m/z 351.40 [M]⁺. HRMS (EI), calcd for C₂₅H₂₁NO 351.1623, found [M]⁺ 351.1618.

### EXAMPLE

N-Methoxycarbonyl-(R)-(+)-1-(2-naphthyl)ethylamine (19). To a solution of (R)-(+)-1-(2-naphthyl)ethylamine 18 (200 mg, 1.2 mmol) in a mixture (1:1) of dioxane and H₂O was added potassium carbonate (323 mg, 2.3 mmol) and methyl chloroformate (0.11 mL, 1.4 mmol) at 0° C. and it was allowed to stir for 1 h at 0° C. The reaction was quenched with 10% HCl solution and extracted with EtOAc. The organic layers were dried over Na₂SO₄ and concentrated under reduced pressure. The residue was purified by silica gel column chromatography to furnish compound 19 (268 mg, >99%) as a colorless oil, R_{f}0.36 (hexane:EtOAc=3:1), [α]20D+96.8 (c=1, CHCl₃). ¹H NMR (300 MHz, CDCl₃): δ 7.82-7.74 (m, 4H), 7.50-7.40 (m, 3H), 5.14 (bm, 1H), 5.00 (bm, 1H), 3.66 (s, 3H), 1.54 (d, 3H, J=67.9 Hz). ¹³C NMR (75 MHz, CDCl₃): δ 156.2, 140.9, 133.1, 132.5, 128.1, 127.7, 127.4, 125.9, 125.5, 124.2, 124.1, 51.8, 50.5, 22.0. MS (EI): m/z 229 [M]⁺. HRMS (EI), calcd for C₁₄H₁₅NO₂ 229.1103, found [M]⁺ 229.1103.

### EXAMPLE

N-Methyl-(R)-(+)-1-(2-naphthyl)ethylamine (20). To a suspension of lithium aluminum hydride (93 mg, 2.4 mmol) in THF (6 mL) was added dropwise a solution of carbamate 19 (268 mg, 1.2 mmol) in THF (1 mL) at 0° C. under argon atmosphere and it was allowed to stir for 1 h at reflux temperature. The reaction was quenched with 1 M NaOH solution at 0° C. and the mixture was filtered through celite pad. The filtrate was concentrated under reduced pressure and the residue was purified by silica gel column chromatography to give amine 20 (186 mg, 86%) as a colorless oil, R_{f}0.21 (CH₂Cl₂:MeOH=9:1), [α]^{20D}+58.0 (c=1, CHCl₃). ¹H NMR (300 MHz, CDCl₃): δ 7.83-7.80 (m, 3H), 7.73 (s, 1H), 7.49-7.40 (m, 3H), 3.81 (q, 1H, J=6.6 Hz), 2.33 (s, 3H), 1.80 (bs, 1H), 1.43 (d, 3H, J=6.6 Hz). ¹³C NMR (75 MHz, CDCl₃): δ 142.4, 133.2, 132.6, 128.0, 127.5, 127.4, 125.7, 125.3, 125.1, 124.6, 60.1, 34.3, 23.7. MS (EI): m/z 185.30 [M]⁺. HRMS (EI), calcd for C₁₃H₁₅N 185.1204, found [M]⁺ 185.1205.

### EXAMPLE

2,N-Dimethyl-N—[(R)-1-(2-naphthyl)ethyl]benzamide (21). The title compound was obtained as described in the general procedure in 87% yield (white solid). R_{f}=0.26 (hexane:EtOAc=3:1), [α]^{20D}+189.1 (c=1, CHCl₃). ¹HNMR(300 MHz, CDCl₃): δ 7.96-7.91 (m, 3.6H), 7.74-7.71 (m, 0.4H), 7.65-7.55 (m, 2.6H), 7.47-7.24 (m, 4.4H), 6.53 (q, 0.6H, J=7.2 Hz), 5.11-5.08 (m, 0.4H), 3.04 (s, 0.7H), 2.97 (s, 0.4H), 2.54 (s, 2.6H), 2.43 (s, 2.3H), 1.82 (d, 2.1H, J=7.2 Hz), 1.79-1.72 (m, 0.9H). ¹³C NMR (75 MHz, CDCl₃): δ 171.5, 137.7, 137.0, 133.6, 133.1, 132.7, 130.3, 128.7, 128.3, 127.9, 127.5, 126.4, 126.2, 126.1, 126.0, 125.6, 125.5, 125.0, 124.6, 56.6, 56.3, 50.0, 30.4, 27.5, 18.9, 18.1, 15.3. MS (EI): m/z 303.30 [M]⁺. HRMS (EI), calcd for C₂₁H₂₁NO 303.1623, found [M]⁺ 303.1627.

### EXAMPLE

4-N⁰-tert-Butoxycarbonylamino-N—[(R)-1-(1-naphthyl)ethyl]-benzamide (22). The title compound was obtained as described in the general procedure in >99% yield (white solid). R_{f}=0.73 (CH₂Cl₂:MeOH=9:1), [α]^{20D}−121.7 (c=1, CHCl₃:MeOH=1:1). ¹H NMR (300 MHz, CDCl₃): δ 8.10 (d, 1H, J=8.1 Hz), 7.80-7.73 (m, 2H), 7.59 (d, 2H, J=8.1 Hz), 7.55 (d, 1H, J=7.5 Hz), 7.49-7.34 (m, 2H), 7.42 (d, 1H, J=7.5 Hz), 7.31 (d, 2H, J=8.1 Hz), 7.04 (s, 1H), 6.52 (d, 2H, J=7.8 Hz), 6.10-6.01 (m, 1H), 1.71 (d, 3H, J=6.6 Hz), 1.47 (s, 9H). ¹³C NMR (75 MHz, CDCl₃): δ 165.8, 152.4, 141.5, 138.3, 133.8, 131.1, 128.6, 128.3, 128.3, 127.9, 126.5, 125.7, 125.1, 123.4, 122.6, 117.6, 80.8, 45.1, 28.2, 20.7. MS (EI): m/z 390.25 [M]⁺. HRMS (EI), calcd for C₂₄H₂₆N₂O₃ 390.1943, found [M]⁺ 390.1947.

### EXAMPLE

4-Amino-N—[(R)-1-(1-naphthyl)ethyl]benzamide (23). The title compound was obtained as described in the compound 9 in 95% yield (slightly yellow solid). R_{f}=0.65 (CH₂Cl₂:MeOH=4:1), [α]^{20D}−137.8 (c=1, CHCl₃:MeOH=4:1). ¹HNMR (300 MHz, CDCl₃): δ 8.15 (d, 1H, J=7.5 Hz), 7.86-7.83 (m, 1H), 7.79 (d, 1H, J=8.1 Hz), 7.58-7.42 (m, 6H), 6.59 (d, 1H, J=8.1 Hz), 6.17 (d, 2H, J=7.5 Hz), 6.13-6.03 (m, 1H), 1.74 (d, 3H, J=6.6 Hz). MS (ED): m/z 290.35 [M]⁺. HRMS (EI), calcd for C₁₉H₁₈N₂O 290.1419, found [M]⁺ 290.142

### EXAMPLE

5-Amino-2-methyl-N—[(R)-1-(1-naphthyl)ethyl]benzamide (24). To a stirred solution of nitro 5i (37 mg, 0.11 mmol) in EtOAc/MeOH (1:1) (3 mL) was added 5% Pd—C (4 mg) and it was allowed to stir for 15 h at 23° C. under H₂ atmosphere. The reaction was filtered through a celite pad and the filtrate was concentrated under reduced pressure. The residue was purified by silica gel column chromatography to furnish compound 24 (27 mg, 80%) as a white solid, R_{f}=0.29 (hexane:EtOAc=1:1), [α]^{20D}−76.8 (c=1, CHCl₃). ¹HNMR(300 MHz, CDCl₃): δ 8.20 (d, 1H, J=8.4 Hz), 7.85 (d, 1H, J=8.0 Hz), 7.78 (d, 1H, J=8.0 Hz), 7.57-7.40 (m, 4H), 6.89 (d, 1H, J=8.0 Hz), 6.70 (bd, 2H, J=13.5 Hz), 6.10-6.07 (bm, 2H), 3.25 (bs, 2H), 2.27 (s, 3H), 1.73 (d, 3H, J=6.0 Hz). ¹³C NMR (75 MHz, CDCl₃): δ 169.0, 143.9, 138.0, 136.9, 133.8, 131.7, 131.1, 128.7, 128.3, 127.2, 126.5, 125.8, 125.1, 123.5, 122.5, 116.6, 113.3, 44.7, 20.5, 18.6. MS (EI): m/z 304.30 [M]⁺.

### EXAMPLE

5-N-Acetylamino-2-methyl-N—[(R)-1-(1-naphthyl)ethyl]benzamide (25). To a stirred solution of amine 24 (14 mg, 0.05 mmol) in CH₂Cl₂ (0.5 mL) was added dropwise triethylamine (9.6 μL, 0.07 mmol) and acetic anhydride (5.2 μL, 0.06 mmol) at 0° C. and it was allowed to stir for 18 h at 23° C. The reaction was quenched with saturated NH₄Cl solution and extracted with CH₂Cl₂. The organic layers were dried over Na₂SO₄ and concentrated under reduced pressure. The residue was purified by silica gel column chromatography to furnish compound 25 (5.4 mg, 34%) as a white solid, R_{f}=0.60 (CH₂Cl₂:MeOH=9:1). ¹HNMR(300 MHz, CDCl₃): δ 8.19 (d, 1H, J=8.1 Hz), 7.85 (d, 1H, J=7.5 Hz), 7.77 (d, 1H, J=8.1 Hz), 7.56-7.39 (m, 4H), 7.35-7.32 (m, 2H), 7.04 (d, 1H, J=7.5 Hz), 6.22 (d, 1H, J=8.1 Hz), 6.12-6.03 (m, 1H), 2.33 (s, 3H), 2.05 (s, 3H), 1.74 (d, 3H, J=6.6 Hz). MS (SI): m/z 346.30 [M]⁺. HRMS (EI), calcd for C₂₂H₂₂N₂O₂ 346.1681, found [M]⁺ 346.1682.

### EXAMPLE

1-Methyl-1-(1-naphthyl)ethylamine (27). CeCl₃-7H₂O(3.77 g, 10.1 mmol) was dried while stirring at 160° C. under reduced pressure for 3 h. Argon was added slowly, and the flash was cooled in an ice bath. THF (20 mL) was added and the suspension was stirred at 23° C. for 2 h. Methyl lithium (1.5 M) in THF (6.7 mL, 10.1 mmol) was added below −50° C. The mixture was stirred for 30 min at −78° C. and a solution of 1-cyanonaphthalene 26 (500 mg, 3.3 mmol) in THF(2 mL) was added. Stirring at 23° C. was continued for 2 h. Conc NH₄OH (6.5 mL) was added at −78° C., and the mixture was warmed to 23° C. and filtered with a celite pad. The solid was washed with CH₂Cl₂. The filtrate was extracted with CH₂Cl₂ and the organic layers were dried over Na₂SO₄ and concentrated under reduced pressure. The residue was taken up in toluene (10 mL) and stirred with 3% H₃PO₄ (10 mL) for 15 min. The toluene layer was extracted with water (×2), and the combined water layers were washed with toluene and made basic with conc. NH₄OH solution. The mixture was extracted with CH₂Cl₂, and the organic layers were dried over Na₂SO₄ and concentrated under reduced pressure to furnish compound 27 (368 mg, 61%) as a colorless oil, R_{f}=0.25 (CH₂Cl₂:MeOH=9:1). ¹H NMR (300 MHz, CDCl₃): δ 9.03 (d, 1H, J=9.0 Hz), 7.98 (d, 1H, J=8.1 Hz), 7.86 (d, 1H, J=8.4 Hz), 7.71 (dd, 1H, J=1.2 and 7.5 Hz), 7.65-7.48 (m, 3H), 1.89 (s, 6H). ¹³C NMR (75 MHz, CDCl₃): δ 144.5, 135.0, 131.2, 129.2, 129.0, 128.1, 127.6, 124.9, 124.8, 122.8, 53.9, 33.3

### EXAMPLE

2-Methyl-5-nitro-N-[1-methyl-1-(1-naphthyl)ethyl]benzamide (28). The title compound was obtained as described in the general procedure in 91% yield (white solid). R_{f}=0.26 (hexane: EtOAc=3:1). ¹H NMR (300 MHz, CDCl₃): δ 8.50 (d, 1H, J=8.1 Hz), 8.07 (d, 1H, J=2.4 Hz), 7.95 (dd, 1H, J=2.4 and 8.4 Hz), 7.87 (d, 1H, J=8.4 Hz), 7.76 (d, 1H, J=8.1 Hz), 7.59 (d, 1H, J=7.5 Hz), 7.54-7.39 (m, 3H), 7.17 (d, 1H, J=8.7 Hz), 6.87 (bs, 1H), 2.24 (s, 3H), 1.95 (s, 6H). ¹³C NMR (75 MHz, CDCl₃): δ 166.2, 145.3, 143.9, 140.6, 138.0, 134.9, 131.3, 131.4, 129.9, 129.8, 128.7, 125.3, 125.2, 125.1, 123.7, 123.7, 121.4, 57.5, 28.5, 19.5.

### EXAMPLE

5-Amino-2-methyl-N-[1-methyl-1-(1-naphthyl)ethyl]benzamide (29). The title compound was obtained as described for compound 24 in 75% yield (slightly yellow solid). R_{f}=0.18 (hexane:EtOAc=1:1). ¹H NMR (400 MHz, CDCl₃): δ 8.56 (d, 1H, J=8.7 Hz), 7.88 (d, 1H, J=7.0 Hz), 7.75 (d, 1H, J=8.1 Hz), 7.65 (d, 1H, J=7.3 Hz), 7.49-7.42 (m, 3H), 6.90 (d, 1H, J=8.0 Hz), 6.60 (s, 1H), 6.53 (d, 1H, J=8.0 Hz), 6.21 (s, 1H), 2.21 (s, 3H), 2.08 (s, 6H), ¹³C NMR (100 MHz, CDCl₃): δ 169.1, 143.9, 141.2, 138.0, 135.0, 131.7, 130.2, 129.7, 128.7, 125.8, 125.2, 125.2, 125.0, 123.7, 116.3, 113.1, 57.5, 28.5, 18.6. MS (EI): m/z 318.45 [M]⁺. HRMS (EI), calcd for C₂₁H₂₂N₂O 318.1732, found [M]⁺ 318.1729.

### EXAMPLE

5-Iodo-2-methylbenzoic Acid (31). NaIO₄ (295 mg, 1.38 mmol) and KI (685 mg, 4.13 mmol) were added over 45 min slowly portionwise to stirred 95% H₂SO₄ (15 mL). Stirring was continued for 1 h at 25-30° C. to give a dark-brown iodinating solution at 25-30° C. To a stirred solution of 2-toluic acid 30 (680 mg, 5 mmol) in 95% H₂SO₄ (5 mL), the iodinating solution was added dropwise over 45 min while maintaining the temperature at 25-30° C. Stirring was continued for 2 h, and the iodination reaction was quenched by slowly pouring the final reaction mixture into stirred ice water. The mixture was extracted with AcOEt and dried over anhydrous Na₂SO₄. The solvent was evaporated under reduced pressure and purification by silica gel flash column chromatography to afford compound 31 in 63% yield. ¹H NMR (400 MHz, CDCl₃): δ 8.38 (d, 1H, J=1.8 Hz), 7.75 (dd, 1H, J=8.1, 1.8 Hz), 7.02 (d, 1H, J=8.1 Hz), 2.59 (s, 3H).

### EXAMPLE

5-Iodo-2-methyl-N-[1-methyl-1-(1-naphthyl)ethyl]benzamide (32). The title compound was obtained as described in the general procedure using DMF:CH₂Cl₂ (1:1) as a solvent in 87% yield (white solid). ¹H NMR (400 MHz, CDCl₃): δ 8.20 (d, 1H, J=8.5 Hz), 7.89 (d, 1H, J=8.0 Hz), 7.83 (d, 1H, J=8.1 Hz), 7.64-7.44 (m, 6H), 6.92 (d, 1H, J=7,8 Hz), 6.12 (m, 1H,), 5.94 (bd, 1H, J=8.3 Hz), 2.36 (s, 3H), 1.80 (d, 3H, J=6.7 Hz). ¹³C NMR (100 MHz, CDCl₃): δ 167.1, 138.6, 138.4, 137.6, 135.6, 135.0, 133.9, 132.7, 131.1, 128.8, 128.6, 126.6, 125.9, 125.1, 123.3, 122.6, 90.0, 44.9, 20.5, 19.3. MS (ESI): m/z 438.0 [M+Na]⁺. HRMS (ESI), calcd for C₂₀H₁₈INONa 438.0331; found [M+Na]⁺ 438.0333.

### EXAMPLE

5-Cyano-2-methyl-N-[1-methyl-1-(1-naphthyl)ethyl]benzamide (33). Compound 32 (29 mg, 0.07 mmol) was dissolved in dry DMF (2 mL). CuCN (62 mg, 0.7 mmol) and a crystal of KCN were added. The mixture was flushed with nitrogen and stirred at 80° C. for 1 h then 130° C. for 10 h. CuCN (62 mg, 0.7 mmol) was added again. The mixture was flushed with nitrogen and stirred at 130° C. for 6 h. After this time, NH₄OH solution was poured into reaction mixture, and the mixture was extracted with AcOEt and dried over anhydrous Na₂SO₄. The solvent was evaporated under reduced pressure and purification by silica gel flash column chromatography to afford compound 33 in 78% yield as a white solid. ¹HNMR(400 MHz, CDCl₃): δ 8.19 (d, 1H, J=8.4 Hz), 7.87 (d, 1H, J=8.3 Hz), 7.84 (d, 1H, J=8.2 Hz), 7.63-7.44 (m, 6H), 7.29 (d, 1H, J=7.8 Hz), 6.12 (m, 1H), 6.08-5.99 (bs, 1H), 2.48 (s, 3H), 1.81 (d, 3H, J=6.6 Hz). ¹³C NMR (100 MHz, CDCl₃): δ 166.6, 141.9, 137.4, 137.2, 133.9, 133.0, 131.8, 131.0, 130.1, 128.9, 128.7, 126.7, 126.0, 125.1, 123.1, 122.6, 118.1, 109.7, 45.0, 20.4, 20.1. MS (EI): m/z 314.10 [M]⁺HRMS(EI), calcd for C₂₁H₁₈N₂O 314.1419, found [M]⁺ 314.1424.

### EXAMPLE

2-Methyl-5-nitrobenzoic Acid Methyl Ester (35). To a stirring MeOH (4 mL) in a round-bottom flask was added dropwise thionyl chloride (0.24 mL, 3.3 mmol) at 0° C. The mixture was added 2-methyl-5-nitrobenzoic acid 34 (300 mg, 1.7 mmol) at 0° C. and it was allowed to stir for 4 h at reflux temperature. The reaction was concentrated under reduced pressure, and the residue was purified by silica gel column chromatography to give corresponding compound 35 (320 mg, 99%) as a colorless oil, R_{f}=0.85 (hexane:EtOAc=1:1). ¹H NMR (400 MHz, CDCl₃): δ 8.52 (s, 1H), 8.05 (s, 1H), 7.30 (s, 1H), 3.83 (s, 3H), 2.56 (s, 3H). ¹³CNMR(100 MHz, CDCl₃): δ 165.4, 147.6, 145.6, 132.5, 130.1, 125.8, 125.3, 52.1, 21.5. MS (EI): m/z 195 [M]⁺. HRMS (EI), calcd for C₉H₉N0₄195.0532, found [M]⁺ 195.0539.

### EXAMPLE

2-Bromomethyl-5-nitrobenzoic Acid Methyl Ester (36). Compound 35 (100 mg, 0.53 mmol) was dissolved in CCl₄ (4 mL), followed by addition of NBS (100 mg, 0.58 mmol) and a catalytic amount of benzoyl peroxide. The mixture was stirred at reflux for 24 h. Another portion, of dibenzoyl peroxide (40 mg, 0.23 mmol) was added and then the mixture was stirred and heated at reflux for another 10 h. The mixture was allowed to cool to 23° C. and was filtered. The filtrate was washed with NaHCO₃, dried over Na₂SO₄, and the solvent evaporated in vacuo. The residue was purified by silica gel column chromatography to afford compound 36 in 89% yield. ¹H NMR (400 MHz, CDCl₃): δ 8.81 (d, 1H, J=2.5 Hz), 8.33 (dd, 1H, J=8.5, 2.5 Hz), 7.68 (d, 1 H, 8.5 Hz), 5.00 (s, 2H), 4.01 (s, 3H).

### EXAMPLE

2-Methoxymethyl-5-nitrobenzoic Acid Methyl Ester (37). NaH (44 mg, 1.1 mmol) was added to a round-bottomed flask containing methanol (2 mL) at 0° C. The sodium methoxide solution was added to a cold solution of compound 36 (60 mg, 0.22 mmol) in methanol (2 mL) at 0° C. The resulting solution was stirred at 50° C. for 4 h. After this time, NH₄Cl solution was poured into reaction mixture at 0° C., and the mixture was extracted with AcOEt and dried over anhydrous Na₂SO₄. The solvent was evaporated under reduced pressure and purification by silica gel flash column chromatography to afford corresponding compound 37 in 72% yield. ¹H NMR (400 MHz, CDCl₃): δ 8.82 (d, 1H, J=2.4 Hz), 8.38 (dd, 1H, J=8.7, 2.4 Hz), 7.94 (d, 1 H, 8.7 Hz), 4.94 (s, 2H), 3.96 (s, 3H), 3.53 (s, 3H).

### EXAMPLE

2-Methoxymethyl-5-nitrobenzoic Acid (38). To a stirring solution of compound 37 (36 mg, 0.75 mmol) in THF/H₂O mixture (5 mL:1 mL) at 0° C. was added solid LiOH.H₂O (120 mg, 5 mmol), and the resulting solution was stirred at 23° C. for 1.5 h. After this period, the reaction mixture was evaporated until 1 mL, and the mixture was extracted with toluene to remove organic impurities. The aqueous layer was cooled to 0° C., acidified with 25% aqueous citric acid until pH 3-4, extracted with AcOEt, and dried over anhydrous Na₂SO₄. The solvent was evaporated and purification by silica gel flash column chromatography to furnish compound 37 in 90% yield. ¹H NMR (400 MHz, CD₃OD and CDCl₃): δ 8.73 (d, 1H, J=2.4 Hz), 8.26 (dd, 1H, J=8.6, 2.4 Hz), 7.80 (d, 1H, 8.6 Hz), 4.88 (s, 2H), 3.43 (s, 3H).

### EXAMPLE

2-Methoxymethyl-5-nitro-N-[1-methyl-1-(1-naphthyl)ethyl]-benzamide (39). The title compound was obtained as described in the general procedure using DMF/CH₂Cl₂ (1:1) as a solvent in 73% yield (white solid). ¹H NMR (400 MHz, CD₃OD and CDCl₃): δ 8.55 (d, 1H, J=2.4 Hz), 8.22 (d, 1H, J=8.4 Hz), 8.21 (d, 1H, 8.4 Hz), 7.90 (d, 1H, J=7.9 Hz), 7.83 (d, 1H, J=8.1 Hz), 7.64-7.46 (m, 5H), 7.43 (br d, J=8.2 Hz), 6.16 (m, 1H), 4.38 and 4.32 (AB, 2H, J=11.5 Hz), 2.92 (s, 3H), 1.81 (d, 3H, J=6.8 Hz).

### EXAMPLE

5-Amino-2-methoxymethyl-N-[1-methyl-1-(1-naphthyl)ethyl]benzamide (40). The title compound was obtained as described for compound 24 in 82% yield (slightly yellow solid). ¹H NMR (400 MHz, CDCl₃): δ 8.24 (d, 1H, J=8.3 Hz), 7.97 (br d, 1H, J=7.7 Hz), 7.88 (d, 1H, J=8.0 Hz), 7.80 (d, 1H, J=8.1 Hz), 7.60-7.43 (m, 4H), 7.15 (d, 1H, J=2.5 Hz), 7.00 (d, 1H, J=8.1 Hz), 6.65 (dd, 1H, J=8.1, 2.5 Hz), 6.15 (m, 1H), 4.08 and 4.02 (AB, 2H, J=10.2 Hz), 3.80 (br s, 2H), 2.70 (s, 3H), 1.77 (d, 3H, J=6.8 Hz). ¹³C NMR (100 MHz, CDCl₃): δ 167.1, 146.9, 138.5, 138.0, 133.9, 132.8, 131.2, 128.7, 128.2, 126.5, 125.8, 125.2, 123.6, 123.2, 122.6, 116.2, 73.2, 56.8, 44.8, 20.4. MS (EI): m/z 334.20 [M]⁺. HRMS (EI), calcd for C₂₁H₂₂N₂O₂ 334.1681, found [M]⁺ 334.1679.

### EXAMPLE

5-Amino-2-methylbenzoic Acid Methyl Ester (41). To a solution of nitro 35 (635 mg, 3.3 mmol) in EtOAc (10 mL) was added 10% Pd—C (30 mg) and it was allowed to stir for 16 h at 23° C. under H₂ atmosphere. The reaction was filtered through a celite pad, and the filtrate was concentrated under reduced pressure. The residue was purified by silica gel column chromatography to furnish compound 41 (536 mg, >99%) as a colorless oil, R_{f}=0.57 (hexane:EtOAc=1:1). ¹HNMR(400 MHz, CDCl₃): δ 7.21 (d, 1H, J=2.5 Hz), 6.97 (d, 1H, J=8.1 Hz), 6.69 (dd, 1H, J=2.5 and 8.1 Hz), 3.82 (s, 3H), 3.64 (s, 2H), 2.43 (s, 3H). ¹³C NMR (100 MHz, CDCl₃): δ 168.1, 144.1, 132.3, 129.8, 129.5, 118.8, 116.7, 51.6, 20.6. MS (EI): m/z 165.20 [M]⁺. HRMS (EI), calcd for C₉H₁₁NO₂165.0790, found [M]⁺ 165.0787.

### EXAMPLE

2-Methyl-5-cyanobenzoic Acid Methyl Ester (42). CuCN (228 mg, 2.5 mmol) was suspended in distilled water (2 mL). NaCN (353 mg, 7.2 mmol) was added with vigorous stirring, and the internal temperature was kept below 40° C. until all the CuCN went into solution. A suspension of amine 41 (350 mg, 2.1 mmol) in water (4 mL) and conc. HCl (0.7 mL) was stirred and cooled in an ice bath. When the temperature reach 5° C., a solution of NaNO₂ (190 mg, 2.8 mmol) in water (0.6 mL) was added dropwise at 5° C. When all the NaNO₂ was added, the solution was added dropwise the NaCN/CuCN solution at 0° C. A few drops of methanol were added to keep the foaming under control. Stirring was continued for 3 h at 23° C. The suspension was extracted with EtOAc, and the organic layers were dried over Na₂SO₄ and concentrated under reduced pressure. The residue was purified by silica gel column chromatography to give compound 42 (115 mg, 31%) as a colorless oil, R_{f}=0.63 (hexane:EtOAc=1:1). ¹H NMR (400 MHz, CDCl₃): δ 8.08 (d, 1H, J=1.7 Hz), 7.56 (dd, 1H, J=1.7 and 7.9 Hz), 7.28 (d, 1H, J=7.9 Hz), 3.83 (s, 3H), 2.56 (s, 3H). ¹³CNMR (100 MHz, CDCl₃): δ 165.7, 145.5, 134.4, 134.1, 132.4, 130.3, 117.8, 109.7, 52.1, 21.8.

### EXAMPLE

5-N-tert-Butoxycarbonylmethylamino-2-methylbenzoic Acid Methyl Ester (43). To a solution of nitrile 42 (40 mg, 0.23 mmol) in MeOH (1.5 mL) was added Boc₂O (0.1 mL, 0.46 mmol) and NiCl₂.6H₂O (5.4 mg, 0.022 mmol) at 0° C. NaBH₄ (61 mg, 1.6 mmol) was then added in small portions over 15 min. The reaction was allowed to stir for 2 h at 23° C. At this point, diethylenetriamine (25 μL, 0.23 mmol) was added. The mixture was allowed to stir for 15 min. The solvent was removed, and the residue was dissolved with EtOAc. The organic layer was washed with saturated NaHCO₃ solution and dried over Na₂SO₄. The solvent was removed under reduced pressure to give a residue, which was purified by silica gel column chromatography to furnish compound 43 (54 mg, 85%) as a colorless oil, R_{f}=0.49 (hexane:EtOAc=3:1). ¹H NMR (400 MHz, CDCl₃): δ 7.78 (s, 1H), 7.29 (d, 1H, J=7.8 Hz), 7.17 (d, 1H, J=7.8 Hz), 4.92 (bs, 1H), 4.26 (d, 2H, J=5.7 Hz), 3.85 (s, 3H), 2.53 (s, 3H), 1.42 (s, 9H). ¹³CNMR(100 MHz, CDCl₃): δ 167.8, 155.8, 139.2, 136.5, 132.0, 131.3, 129.4, 129.5, 79.5, 51.8, 44.0, 28.3, 21.3. MS (CI): m/z 278.30 [M]⁺. HRMS (CI), calcd for C₁₅H₂₀NO₄ 278.1392, found [M−H]⁺ 278.1398.

### EXAMPLE

5-(N,N-tert-Butoxycarbonylmethyl)methylamino-2-methylbenzoic Acid Methyl Ester (44). To a solution of N-Boc amine 43 (60 mg, 0.21 mmol) in THF (3 mL) was added dropwise 0.5 M KHMDS in toluene (0.64 mL, 0.32 mmol) at 0° C. under argon atmosphere and it was allowed to stir for 30 min at 0° C. The mixture was added dropwise MeI (21 μL, 0.34 mmol) at 0° C. and it was allowed to stir for 16 h at 23° C. The reaction was quenched with saturated NH₄Cl solution and extracted with EtOAc. The organic layers were dried over Na₂SO₄ and concentrated under reduced pressure. The residue was purified by silica gel column chromatography to give compound 44 (52 mg, 83%) as a colorless oil, R_{f}=0.60 (hexane:EtOAc=3:1). ¹H NMR (400 MHz, CDCl₃): 6 7.75 (s, 1H), 7.24 (bs, 1H), 7.17 (d, 1H, J=7.8 Hz), 4.36 (bs, 2H), 3.85 (s, 3H), 2.80 and 2.74 (each s, 3H), 2.54 (s, 3H), 1.45 (s, 9H). ¹³C NMR (100 MHz, CDCl₃): δ 167.8, 155.6, 139.1, 135.6, 131.9, 131.2, 130.8, 129.5, 79.8, 51.7, 33.8, 28.3, 21.3.

### EXAMPLE

5-N-tert-Butoxycarbonylmethylamino-2-methylbenzoic Acid (45). To a solution of ester 43 (54 mg, 0.19 mmol) in a mixture (9:1) of THF and water (2 mL) was added LiOH—H₂O (12 mg, 0.29 mmol) at 0° C. and it was allowed to stir for 16 h at 23° C. The reaction was concentrated under reduced pressure, and the residue was diluted with saturated NaHCO₃ solution. The mixture was extracted with Et₂O, and the aqueous layer was acidified with 1 M HCl solution to pH 4. The white solid was extracted with EtOAc, and the organic layers were dried over Na₂SO₄, and concentrated under reduced pressure to provide corresponding acid 45 (39 mg, 76%) as a white solid, R_{f}=0.51 (CH₂Cl₂:MeOH=9:1). ¹H NMR (300 MHz, CDCl₃): δ 7.78 (s, 1H), 7.27 (d, 1H, J=7.8 Hz), 7.15 (d, 1H, J=7.8 Hz), 4.78 (bs, 1H), 4.19 (s, 2H), 2.50 (s, 3H), 1.40 (s, 9H). ¹³CNMR (75 MHz, CDCl₃): δ 170.8, 158.0, 139.5, 135.3, 132.5, 131.4, 130.2, 129.7, 80.1, 44.2, 28.7, 21.6.

### EXAMPLE

5-N-tert-Butoxycarbonylmethylamino-2-methyl-N⁰—[(R)-1-(1-naphthyl)ethyl]benzamide (47). The title compound was obtained as described in the general procedure in 91% yield (white solid). R_{f}=0.20 (hexane:EtOAc=3:1). ¹H NMR (300 MHz, CDCl₃): δ 8.20 (d, 1H, J=8.4 Hz), 7.85 (d, 1H, J=7.5 Hz), 7.78 (d, 1H, J=8.4 Hz), 7.58-7.40 (m, 4H), 7.13 (d, 2H, J=7.5 Hz), 7.08 (d, 1H, J=7.8 Hz), 6.15-6.06 (bm, 2H), 4.86 (bs, 1H), 4.14 (d, 2H, J=5.1 Hz), 2.36 (s, 3H), 1.75 (d, 3H, J=6.0 Hz), 1.39 (s, 9H). ¹³C NMR (75 MHz, CDCl₃): δ 168.7, 155.8, 137.9, 136.5, 136.5, 134.9, 133.9, 131.1, 128.7, 128.7, 128.4, 127.2, 126.5, 125.9, 125.5, 125.1, 123.5, 122.5, 79.5, 44.8, 43.9, 28.3, 20.6, 19.3. MS (EI): m/z 418.45 [M]⁺. HRMS (EI), calcd for C₂₆H₃₀N₂O₃ 418.2256, found [M]⁺ 418.2252.

### EXAMPLE

5-(N,N-tert-Butoxycarbonylmethyl)methylamino-2-methyl-N⁰—[(R)-1-(1-naphthyl)ethyl]benzamide (48). The title compound was obtained as described for compound 45 and general procedure in 98% yield as two steps (white solid). R_{f}=0.20 (hexane:EtOAc=3:1), [α]^{20D}−46.3 (c=1, CHCl₃). ¹H NMR(300 MHz, CDCl₃): δ 8.22 (d, 1H, J=8.1 Hz), 7.85 (d, 1H, J=7.5 Hz), 7.78 (d, 1H, J=8.4 Hz), 7.58-7.40 (m, 4H), 7.11 (d, 1H), J=7.2 Hz), 6.16-6.05 (m, 1H), 6.04 (bs, 1H), 4.28 (s, 2H), 2.71 (s, 3H), 2.38 (s, 3H), 1.76 (d, 3H, J=6.3 Hz), 1.39 (s, 9H). ¹³CNMR(75 MHz, CDCl₃): δ 168.8, 155.5, 137.8, 136.6, 135.6, 134.9, 133.0, 131.1, 128.8, 128.7, 128.4, 127.2, 126.5, 125.9, 125.6, 125.1, 123.5, 122.5, 79.6, 51.9, 44.7, 33.8, 28.3, 20.5, 19.4. MS (ESI): m/z 455.99 [M+Na]⁺. HRMS (ESI), calcd for C₂₇H₃₂N₂O₃Na 455.2311, found [M +Na]⁺ 455.2312.

### EXAMPLE

N-Methyl-5-methylamino-2-methyl-N⁰—[(R)-1-(1-naphthyl)ethyl]benzamide (49). The title compound was obtained as described for compound 9 in 76% yield (white solid). R_{f}=0.27 (CH₂Cl₂:MeOH=9:1), [α]^{20 D}−71.5 (c=1, MeOH). ¹H NMR (300 MHz, CDCl₃ plus a small amount of CD₃OD): δ 8.25 (d, 1H, J=8.1 Hz), 7.88 (d, 1H, J=8.4 Hz), 7.79 (d, 1H, J=8.4 Hz), 7.63 (d, 1H, J=7.3 Hz), 7.59-7.44 (m, 3H), 7.25 (d, 2H, J=7.8 Hz), 7.17 (d, 1H, J=7.8 Hz), 6.05 (q, 1H, J=6.9 Hz), 3.61 (s, 2H), 2.32 (s, 3H), 2.31 (s, 3H), 1.69 (d, 3H, J=6.9 Hz). ¹³C NMR (75 MHz, CDCl₃ plus a small amount of CD₃OD): δ 171.9, 140.3, 138.1, 137.8, 135.7, 135.5, 132.4, 131.8, 130.9, 129.9, 129.0, 128.2, 127.3, 126.7, 126.5, 124.3, 123.8, 55.7, 46.3, 35.5, 21.4, 19.4. MS (EI): m/z 332.30 [M]⁺. HRMS (EI), calcd for C₂₂H₂₄N₂O 332.1889, found [M]⁺ 332.1891.

### EXAMPLE

5-Methylamino-2-methyl-N—[(R)-1-(1-naphthyl)ethyl]benzamide (2). The title compound was obtained as described for compound 9 in 56% yield (white solid). R_{f}=0.11 (CH₂Cl₂: MeOH=9:1). ¹HNMR (400 MHz, CDCl₃ plus a small amount of CD₃OD): δ 8.14 (d, 1H, J=8.5 Hz), 7.78 (d, 1H, J=8.0 Hz), 7.69 (d, 1H, J=8.2 Hz), 7.52 (d, 1H, J=7.1 Hz), 7.47-7.34 (m, 3H), 7.16-7.15 (m, 2H), 7.06 (d, 1H, J=8.2 Hz), 5.93 (q, 1H, J=6.8 Hz), 3.61 (s, 2H), 2.21 (s, 3H), 1.59 (d, 3H, J=6.8 Hz). ¹³C NMR (100 MHz, CDCl₃ plus a small amount of CD₃OD): δ 172.0, 140.9, 140.3, 138.1, 135.5, 135.3, 132.4, 131.9, 129.9, 130.0, 129.0, 127.3, 127.1, 126.7, 126.5, 124.3, 123.7, 46.3, 46.0, 21.4, 19.3. MS (EI): m/z 318.30 [M]⁺. HRMS (EI), calcd for C₂₁H₂₂N₂O 318.1732, found [M]⁺ 318.1734.

1. A compound of formula 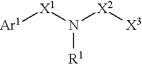
or a pharmaceutically acceptable salt thereof, is described wherein
Ar¹ is 1-napthyl, quinolinyl, isoquinolinyl, or quinazolinyl, each of which is optionally substituted;
X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, arylalkyl and heteroarylalkyl, each of which is optionally substituted; or R³ and R⁴ are taken together with the attached carbon to form a cycloalkylene;
R¹ is hydrogen, alkyl, arylalkyl, heteroarylalkyl, hydroxyl, alkoxyl or a pro-drug moiety, each of which is optionally substituted; and X² is selected from the group consisting of a bond, alkylene and heteroalkylene, or R¹ and X² are taken together with the attached nitrogen to form an optionally substituted heterocycle; and
X³ is an acyl, a carboxylate, or a derivative thereof, a sulfonate, or a sulfonamide group;
providing that when X¹ is CR³R⁴, the absolute stereochemistry is (R); and
providing that the compound does not have the formula:

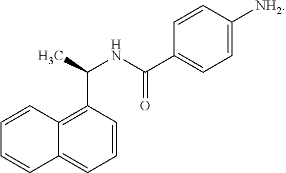

2. A compound of formula 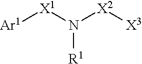
or a pharmaceutically acceptable salt thereof, is described wherein
Ar¹ is optionally substituted 2-napthyl;
X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; R³ and R⁴ are in each instance independently selected from the group consisting of hydrogen, alkyl, alkoxyl, arylalkyl and heteroarylalkyl, each of which is optionally substituted; or R³ and R⁴ are taken together with the attached carbon to form a cycloalkylene;
R¹ is hydrogen, alkyl, arylalkyl, heteroarylalkyl, hydroxyl, alkoxyl or a pro-drug moiety, each of which is optionally substituted;
X² is selected from the group consisting of a bond, alkylene and heteroalkylene, or
R¹ and X² are taken together with the attached nitrogen to form an optionally substituted heterocycle; and
X³ is an acyl, a carboxylate, or a derivative thereof, a sulfonate, or a sulfonamide group; and
providing that when X¹ is CR³R⁴, the absolute stereochemistry is (R); and providing that when X¹ CH(CH₃), R¹ is hydrogen, X² is a bond, and X³ is optionally substituted benzoyl, then X³ includes at least one hydrogen containing hydrogen-bonding group.

3. (canceled)

4. The compound of claim 1 of the formula 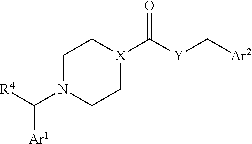
or a pharmaceutically acceptable salt thereof, is described wherein
Ar¹ is aryl or heteroaryl, each of which is optionally substituted;
Ar² is aryl or heteroaryl, each of which is optionally substituted;
R⁴ is hydrogen, alkyl, alkoxyl, arylalkyl or heteroarylalkyl, each of which is optionally substituted;
Y is N(R^{1A}) or O; where R^{1A} is hydrogen, alkyl, arylalkyl, heteroarylalkyl, hydroxyl, alkoxyl or a pro-drug moiety, each of which is optionally substituted; and
X is CH or N.

5. The compound of claim 4 wherein Ar' is bicyclic aryl or bicyclic heteroaryl, each of which is optionally substituted.

6. The compound of claim 4 wherein Ar² is optionally substituted phenyl.

7. The compound of claim 4 wherein Y is N(R^{1A}), where R^{1A} is hydrogen.

8. The compound of claim 4 of the formula 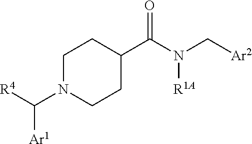
or a pharmaceutically acceptable salt thereof, is described wherein
Ar¹ is aryl or heteroaryl, each of which is optionally substituted;
Ar² is optionally substituted phenyl;
R^{1A} is hydrogen, alkyl, arylalkyl, heteroarylalkyl, hydroxyl, alkoxyl or a pro-drug moiety, each of which is optionally substituted; and
R⁴ is hydrogen, alkyl, alkoxyl, arylalkyl or heteroarylalkyl, each of which is optionally substituted.

9. The compound of claim 8 wherein Ar¹ is bicyclic aryl or bicyclic heteroaryl, each of which is optionally substituted.

10. The compound of claim 8 wherein Ar¹ is selected from the group consisting of 1-naphthyl, 2-naphthyl, 4-quinolinyl, 4-isoquinolinyl and 4-quinazolinyl, each of which is optionally substituted

11. The compound of claim 8 wherein Ar² is monocyclic aryl or monocyclic heteroaryl, each of which is optionally substituted.

12. The compound of claim 8 wherein Ar² is optionally substituted phenyl.

13. The compound of claim 8 wherein Ar² is optionally substituted pyrdinyl.

14. The compound of claim 8 wherein Ar² is optionally substituted thienyl.

15. The compound of claim 8 wherein R^{IA} is hydrogen.

16. The compound of claim 4 or wherein R⁴ is optionally substituted alkyl.

17. A method for treating a patient in need of relief from a respiratory viral infection; the method comprising the step of administering to the patient a therapeutically effective amount of a compound, or a composition comprising the compound, where the compound is of the formula 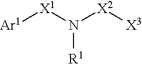
or a pharmaceutically acceptable salt thereof, wherein
Ar¹ is aryl or heteroaryl, each of which is optionally substituted;
X¹ is NR² or CR³R⁴, wherein R² is selected from the group consisting of hydrogen, alkyl, arylalkyl, heteroarylalkyl, hydroxyl, alkoxyl and a pro-drug moiety, each of which is optionally substituted; and R³ and R⁴ are in each instance independently selected from the group consisting of hydrogen, alkyl, alkoxy, arylalkyl and heteroarylalkyl, each of which is optionally substituted; or R³ and R⁴ are taken together with the attached carbon to form a cycloalkylene;
R¹ is hydrogen, alkyl, arylalkyl, heteroarylalkyl, hydroxyl, alkoxyl or a pro-drug moiety, each of which is optionally substituted; and X² is selected from the group consisting of a bond, alkylene and heteroalkylene, or R¹ and X² are taken together with the attached nitrogen to form an optionally substituted heterocycle; and
X³ is an acyl, a carboxylate or a derivative thereof, a sulfonate, or a sulfonamide group.

18.-42. (canceled)
